(19) United States
(12) Patent Application Publication
(10) Pub. No.: US 2025/0134654 A1
(43) Pub. Date: May 1, 2025

(54) VALVE-IN-VALVE REPLACEMENT HEART VALVE PROSTHESIS

(71) Applicant: Medtronic, Inc., Minneapolis, MN (US)

(72) Inventors: David A. Grossman, Santa Rosa, CA (US); Karina Sanchez, Morgan Hill, CA (US); Tasha Ann Cheshko Meziere, Irvine, CA (US); Brianna L. Beenken, St. Louis Park, MN (US); Chirag R. Gajjar, Rohnert Park, CA (US)

(73) Assignee: Medtronic, Inc., Minneapolis, MN (US)

(21) Appl. No.: 18/921,526

(22) Filed: Oct. 21, 2024

Related U.S. Application Data
(60) Provisional application No. 63/593,797, filed on Oct. 27, 2023.

(51) Int. Cl. A61F 2/24

(52) U.S. Cl. CPC A61F 2/2418; A61F 2/2436; A61F 2220/0008

(57) ABSTRACT

A heart valve prosthesis includes a frame defining a central lumen, a valve assembly disposed within the central lumen of the frame, and a plurality of gripper pads. The gripper pads are configured to engage lacerated leaflets of a previously implanted heart valve prosthesis. The gripper pads are configured to separate from each other to separate the lacerated leaflets when the heart valve prosthesis radially expands from a radially compressed configuration to a radially expanded configuration.

### CROSS-REFERENCE TO RELATED APPLICATIONS

This application claims the benefit of U.S. Provisional Application No. 63/593,797, filed Oct. 27, 2023, the contents of which are incorporated by reference herein in their entirety.

### FIELD

The present technology is related generally to replacement prosthetic valve devices, and more specifically to leaflet gripper pads on a replacement heart valve prosthesis for valve-in-valve replacement.

### BACKGROUND

The human heart is a four-chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs to the remaining body. To ensure that blood flows in one direction through the heart, atrioventricular valves (tricuspid and mitral valves) are present between the junctions of the atria and the ventricles, and semi-lunar valves (pulmonary valve and aortic valve) govern the exits of the ventricles leading to the lungs and the rest of the body. These valves contain valve leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots, which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based systems. Heart valve prostheses can be delivered while in a low profile or compressed/collapsed arrangement so that the heart valve prosthesis can be advanced through the patient's vasculature. Once positioned at the treatment site, the heart valve prosthesis can be expanded to engage tissue at the diseased heart valve region to, for instance, hold the heart valve prosthesis in position.

A previously implanted heart valve prosthesis may require replacement for a variety of reasons. A valve-in-valve replacement of a previously implanted heart valve prosthesis with a replacement heart valve prosthesis (i.e., deploying the replacement heart valve prosthesis within the previously implanted heart valve prosthesis) may increase the risks of coronary artery obstruction, sinus sequestration, and restriction of the coronary perfusion pathway in patients. Accordingly, there remains a need for improved valve-in-valve heart valve prostheses designs and methods that reduce the risks of coronary artery obstruction and sinus sequestration.

### BRIEF SUMMARY

The techniques and devices of this disclosure generally relate to heart valve prostheses with leaflet gripper pads. The leaflet gripper pads are configured to engage lacerated valve leaflets of a failed or failing previously implanted heart valve prosthesis when the replacement heart valve prosthesis is in a radially compressed configuration. As the replacement heart valve prosthesis expands to a radially expanded configuration for deployment, the lacerated valve leaflets of the previously implanted heart valve prosthesis expand or diverge to provide a coronary profusion pathway.

In an example hereof, the present disclosure relates to a heart valve prosthesis having a radially expanded configuration and a radially compressed configuration. The heart valve prosthesis comprises a frame, the frame defining a central lumen, a valve assembly disposed within the central lumen of the frame, and a plurality of gripper pads. Each gripper pad is configured to engage lacerated leaflets of a previously implanted heart valve prosthesis or a of a native heart valve. The plurality of gripper pads are configured to separate from each other to separate the lacerated leaflets of the previously implanted heart valve prosthesis or of the native heart valve when the heart valve prosthesis radially expands from the radially compressed configuration to the radially expanded configuration.

In another example hereof, in the heart valve prosthesis according to any of the preceding or following examples, the plurality of gripper pads are coupled to an outer surface of the frame.

In another example hereof, in the heart valve prosthesis according to any of the preceding or following examples, the heart valve prosthesis further comprises an outer skirt coupled to an outer surface of the frame, and wherein the plurality of gripper pads are coupled to an outer surface of the outer skirt.

In another example hereof, in the heart valve prosthesis according to any of the preceding or following examples, the plurality of gripper pads includes at least one gripper pad pair including a first gripper pad and a second gripper pad.

In another example hereof, in the heart valve prosthesis according to any of the preceding or following examples, the first gripper pad and the second gripper pad are disposed adjacent each other between a first commissure of the valve assembly and a second commissure of the valve assembly.

In another example hereof, in the heart valve prosthesis according to any of the preceding or following examples, the first gripper pad is spaced a first circumferential distance from the second gripper pad in the radially compressed configuration and the first gripper pad is spaced a second distance from the second gripper pad in the radially expanded configuration, wherein the second distance is greater than the first distance.

In another example hereof, in the heart valve prosthesis according to any of the preceding or following examples, the plurality of gripper pads are substantially parallel to a central longitudinal axis of the heart valve prosthesis.

In another example hereof, in the heart valve prosthesis according to any of the preceding or following examples, the plurality of gripper pads are angled with respect to a central longitudinal axis of the heart valve prosthesis.

In another example hereof, in the heart valve prosthesis according to any of the preceding or following examples, the first gripper pad and the second gripper pad are angled with respect to a frame longitudinal axis of the frame disposed between the first gripper pad and the second gripper pad, wherein the longitudinal axis is substantially parallel to the central longitudinal axis of the heart valve prosthesis.

In another example hereof, in the heart valve prosthesis according to any of the preceding or following examples, a first longitudinal axis of the first gripper pad and a second longitudinal axis of the second gripper pad are at a first angle with respect to the frame longitudinal axis in the radially compressed configuration, and wherein the first longitudinal axis of the first gripper pad and a second longitudinal axis of the second gripper pad are at a second angle with respect to the frame longitudinal axis in the radially expanded configuration, wherein the second angle is greater than the first angle.

In another example hereof, in the heart valve prosthesis according to any of the preceding or following examples, the plurality of gripper pads comprises two gripper pad pairs, each of the two gripper pad pairs comprising two gripper pads.

In another example hereof, in the heart valve prosthesis according to any of the preceding or following examples, the lacerated leaflets are of the previously implanted heart valve prosthesis.

In another example hereof, in the heart valve prosthesis according to any of the preceding or following examples, the lacerated leaflets are of the native heart valve.

In another example hereof, the present disclosure is related to a delivery system for delivering a heart valve prosthesis. The delivery system comprises an outer shaft including an outer shaft lumen, an inner shaft disposed within the outer shaft lumen. a balloon coupled to the inner shaft, a heart valve prosthesis mounted on the expandable balloon, and a leaflet clip mechanism. The heart valve prosthesis includes a frame, a valve assembly, and a plurality of gripper pads disposed on an outer surface of the heart valve prosthesis. The leaflet clip mechanism is configured to selectively impart an inward radial force on a plurality of valve leaflets of a previously implanted heart valve prosthesis or of a native heart valve such that a valve leaflet of the plurality of leaflets is engaged with a gripper pad of the plurality of gripper pads.

In another example hereof, in the delivery system according to any of the preceding or following examples, the leaflet clip mechanism comprises a first shaft and a plurality of fingers extending distally from the first shaft, the plurality of fingers configured to engage and outer surface of the plurality of valve leaflets of the previously implanted heart valve prosthesis or of the native heart valve.

In another example hereof, in the delivery system according to any of the preceding or following examples, the leaflet clip mechanism further comprises a second shaft and a tension loop extending from a distal end of the second shaft, wherein the second shaft is disposed radially outside the first shaft and the tension loop is disposed radially outside the plurality of fingers, wherein the tension loop is configured to impart a radially inward force on the plurality of fingers.

In another example hereof, in the delivery system according to any of the preceding or following examples, the leaflet clip mechanism comprises a shaft and a tension loop extending from a distal end of the shaft, wherein the tension loop is configured to impart a radially inward force on the plurality of valve leaflets of the previously implanted heart valve prosthesis or of the native heart valve.

In another example hereof, in the delivery system according to any of the preceding or following examples, the lacerated leaflets are of the previously implanted heart valve prosthesis.

In another example hereof, in the delivery system according to any of the preceding or following examples, the lacerated leaflets are of the native heart valve.

In another example hereof, the present disclosure is related to a method of delivering and deploying a heart valve prosthesis. The method comprises: advancing a delivery system including the heart valve prosthesis to a site of a native heart valve, the heart valve prosthesis including a plurality of gripper pads on an outer surface thereof; locating the heart valve prosthesis within lacerated leaflets of the native heart valve or of a previously implanted heart valve prosthesis; advancing a leaflet clip mechanism over the lacerated leaflets; partially expanding the heart valve prosthesis such that a radially outward force is imparted on the gripper pads and a radially inward force is imparted on the lacerated leaflets by the leaflet clip mechanism such that the gripper pads engage with the lacerated leaflets; proximally retracting the leaflet clip mechanism; and fully expanding the heart valve prosthesis such that the gripper pads separate the lacerated leaflets.

In another example hereof, in the method according to any of the preceding or following examples, the delivery system comprises an expandable balloon, wherein the heart valve prosthesis is mounted on the balloon, wherein partially expanding the heart valve prosthesis comprises partially expanding the balloon, and wherein fully expanding heart valve prosthesis comprises further expanding the balloon.

In another example hereof, in the method according to any of the preceding or following examples, the leaflet clip mechanism comprises a first shaft with a plurality of fingers extending from a distal end of the first shaft, wherein advancing the leaflet clip mechanism comprises distally advancing the first shaft to distally advance the plurality of fingers.

In another example hereof, in the method according to any of the preceding or following examples, the plurality of fingers are configured to expand radially outward such that advancing the plurality of figures out of an outer shaft of the delivery system enables the plurality of fingers to expand radially outward and over the lacerated leaflets.

In another example hereof, in the method according to any of the preceding or following examples, the leaflet clip mechanism further comprises a second shaft disposed over the first shaft and a tension loop extending from a distal end of the second shaft, wherein advancing the leaflet clip mechanism further includes advancing the second shaft such that the tension loop imparts a radially inward force to the fingers to impart a radially inward force on the lacerated leaflets.

In another example hereof, the method according to any of the preceding or following examples further comprises rotationally aligning the heart valve prosthesis relative to the native heart valve or the previously implanted heart valve prosthesis, such that the plurality of gripper pads align with the lacerated leaflets.

In another example hereof, in the method according to any of the preceding or following examples, locating the heart valve prosthesis comprises locating the heart valve prosthesis within lacerated leaflets of the native heart valve.

In another example hereof, in the method according to any of the preceding or following examples, locating the heart valve prosthesis comprises locating the heart valve prosthesis within lacerated leaflets of the previously implanted heart valve prosthesis.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings are not to scale.

FIG. 1 depicts a schematic illustration of a side view of a prior art heart valve prosthesis.

FIG. 2 depicts a schematic illustration of a perspective outflow view of the prior art heart valve prosthesis of FIG. 1.

FIG. 3A depicts an illustration of a heart valve prosthesis with gripper pads in a radially compressed configuration according to embodiments hereof.

FIG. 3B depicts an illustration of the heart valve prosthesis of FIG. 3A in a radially expanded configuration.

FIG. 4A depicts an illustration of a heart valve prosthesis in a radially compressed configuration according to embodiments hereof.

FIG. 4B depicts an illustration of the heart valve prosthesis of FIG. 4A in a radially expanded configuration.

FIG. 5A depicts a schematic illustration of a perspective view of the heart valve prosthesis of FIG. 3A or 4A in the radially compressed configuration.

FIG. 5B depicts a schematic illustration of an outflow end view of the heart valve prosthesis of FIG. 3A of 4A in the radially compressed configuration.

FIG. 5C depicts a schematic illustration of a perspective view of the heart valve prosthesis of FIG. 3B or 4B in the radially expanded configuration.

FIG. 5D depicts a schematic illustration of an outflow end view of the heart valve prosthesis of FIG. 3B of 4B in the radially expanded configuration.

FIG. 6A depicts an illustration of a heart valve prosthesis in a radially compressed configuration according to embodiments hereof.

FIG. 6B depicts an illustration of the heart valve prosthesis of FIG. 6A in a radially expanded configuration.

FIG. 7A depicts a schematic illustration of a perspective view of the heart valve prosthesis of FIG. 6A.

FIG. 7B depicts a schematic illustration of an outflow end view of the heart valve prosthesis of FIG. 6A.

FIG. 7C depicts a schematic illustration of a perspective view of the heart valve prosthesis of FIG. 6B.

FIG. 7D depicts a schematic illustration of a cross-section view of the heart valve prosthesis of FIG. 6B.

FIGS. 8A-8G depict illustrations of various embodiments of gripper pad connectors.

FIG. 9 depicts a distal end of a delivery system with a leaflet clip mechanism according to embodiments hereof.

FIG. 10 depicts a perspective view of the delivery system of FIG. 9 with a leaflet clip mechanism according to embodiments hereof.

FIG. 11 depicts a schematic illustration of a perspective view of the delivery system of FIG. 9 with a leaflet clip mechanism according to embodiments hereof.

FIGS. 12-13 depict a schematic illustration of a perspective view of the delivery system of FIG. 9 with a leaflet clip mechanism according to embodiments hereof.

FIG. 14 is a flow chart depicting a method for a valve-in-valve heart valve replacement according to embodiments hereof.

FIGS. 15-24 depict illustrations of several of the steps of the method of FIG. 14 for a valve-in-valve heart valve replacement according to embodiments hereof.

### DETAILED DESCRIPTION OF THE INVENTION

Specific embodiments of the present disclosure are now described with reference to the figures wherein like reference numbers indicate identical or functionally similar elements. The following detailed description describes examples of embodiments of the disclosure and is not intended to limit the present technology or the application and uses of the present technology. Although the description of embodiments hereof is in the context of an aortic heart valve prosthesis, the present technology may also be used in other valve prostheses. For example, embodiments of the gripper pads and delivery systems described herein may be utilized with a heart valve prosthesis configured for placement within a pulmonary, aortic, mitral, or tricuspid valve, or may be utilized with a valve prosthesis configured for placement within a venous valve or within other body passageways where it is deemed useful. Furthermore, there is no intention to be bound by any expressed or implied theory presented in the preceding technical field, background, brief summary, or the following detailed description.

The terms “distal” and “proximal” when used in the following description to refer to a catheter or delivery system are with respect to a position or direction relative to the treating clinician or handle of the delivery system. Thus, “distal” and “distally” refer to positions distant from or in a direction away from the clinician or handle and “proximal” and “proximally” refer to positions near or in a direction toward the clinician or handle. When the terms “distal” and “proximal” are used in the following description to refer to a device to be implanted into a vessel, such a heart valve prosthesis, they are used with reference to the direction of blood flow. Thus, “distal” and “distally” refer to positions in a downstream direction with respect to the direction of blood flow and “proximal” and “proximally” refer to positions in an upstream direction with respect to the direction of blood flow.

The embodiments disclosed herein are directed to heart valve prostheses with gripper pads configured to engage lacerated valve leaflets of a previously implanted heart valve prosthesis or of a native heart valve, delivery systems to be used with the gripper pad equipped heart valve prosthesis, and methods of delivering and deploying gripper pad equipped heart valve prostheses. In embodiments, the gripper pads engage and separate lacerated valve leaflets of the previously implanted heart valve prosthesis or the native heart valve and thereby reduce the risks of coronary artery obstruction, sinus sequestration, and improve coronary perfusion.

When the “lacerated leaflets” of the previously implanted heart valve prosthesis or the native heart valve are discussed herein, the leaflets may be lacerated by any procedure and/or device known to those skilled in the art. For example, and not by way of limitation, U.S. Patent Publication No. 2020/0146690, U.S. Patent Publication No. 2020/0146691, and WIPO Publication No. WO 2023/053063, each assigned to Medtronic, Inc. and incorporated by reference herein in its entirety, describe devices and methods for lacerating leaflets of a native heart valve or of a previously implanted heart valve prosthesis. Similarly, other devices and procedures, such as described in Khan J M, et al., “Transcatheter Laceration of Aortic Leaflets to Prevent Coronary Obstruction During Transcatheter Aortic Valve Replacement: Concept to First-in-Human”, JACC Cardiovasc Interv. 2018 Apr. 9; 11 (7): 677-689, may also be used.

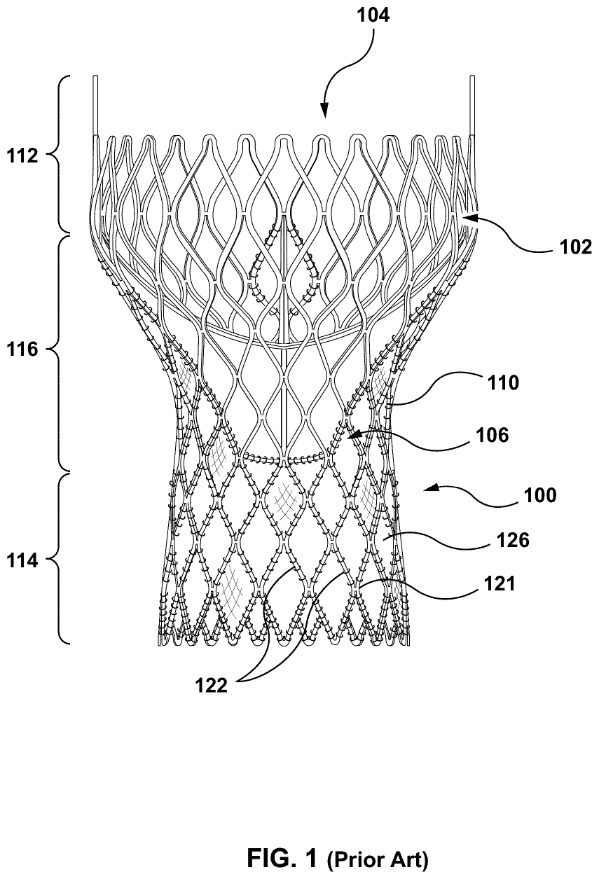

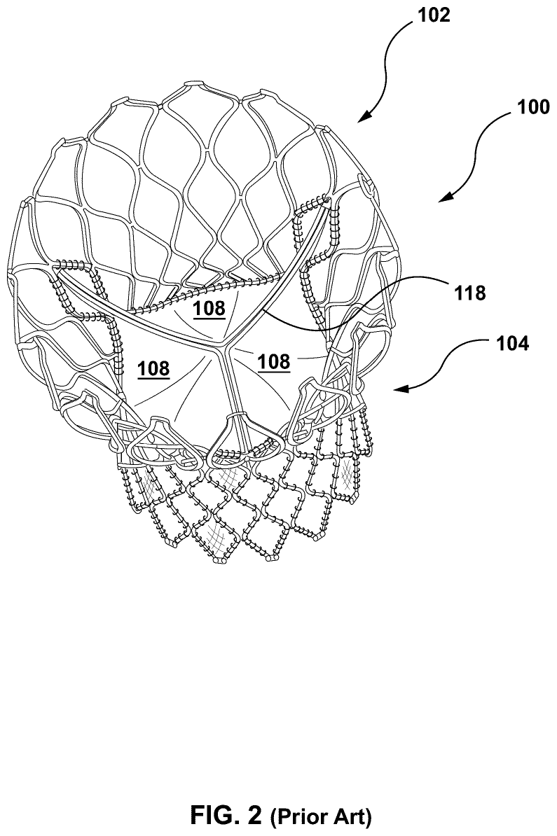

FIGS. 1 and 2 show an exemplary heart valve prosthesis 100 similar to the Medtronic Core Valve® transcatheter aortic valve replacement valve prosthesis and as described in U.S. Patent Application Publication No. 2011/0172765 to Nguyen et al. (hereinafter “the '765 publication”), which is incorporated by reference herein in its entirety. The heart valve prosthesis 100 includes a frame 102 having a substantially tubular structure defining a central lumen 104 therethrough, and a valve assembly 106 affixed to an inner surface of the frame 102. The valve assembly 106 comprises a plurality of valve leaflets 108 coupled to an inner skirt 110. The valve assembly 106 is configured to block flow in one direction to regulate flow therethrough. The valve assembly 106 may be formed from a natural or man-made material, including, but not limited to, mammalian tissue, such as porcine, equine, or bovine pericardium, or a synthetic or polymeric material.

The frame 102 in the exemplary embodiment includes an outflow portion 112, an inflow portion 114, and a transition portion 116 between the inflow portion 114 and the outflow portion 112. The frame 102 may comprise a plurality of struts 122 forming a plurality of side openings 126. The frame 102 has a radially compressed or crimped configuration for delivery and a radially expanded configuration when deployed. The frame 102 may be a self-expanding or balloon expandable structure formed by laser cutting or etching of a metal alloy tube comprising, for example, stainless steel or nickel titanium.

The valve assembly 106 may include the inner skirt 110 affixed to the frame 102, and three (3) valve leaflets 108 configured to block blood flow in one direction to thereby regulate blood flow through the central lumen 104 of the frame 102. The valve assembly 106 may be formed from a natural or man-made material, including, but not limited to, mammalian tissue, such as porcine, equine, or bovine pericardium, or a synthetic or polymeric material.

The heart valve prosthesis 100 described is an example of a heart valve prosthesis that may be a previously implanted heart valve prosthesis as described herein. Therefore, the heart valve prosthesis 100 will be described below as “the previously implanted heart valve prosthesis 100.” However, this is not meant to be limiting, and the “previously implanted heart valve prosthesis” described below need not be the heart valve prosthesis 100, and instead can be any heart valve prosthesis.

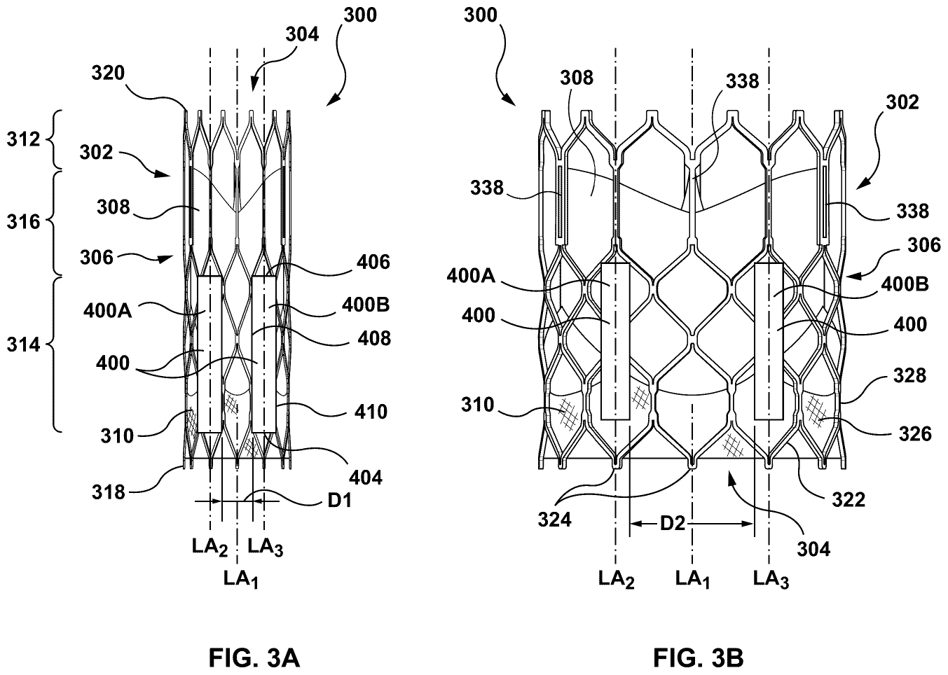

FIGS. 3A and 3B show a heart valve prosthesis 300 according to embodiments herein.

The heart valve prosthesis 300 is configured for deployment as a valve-in-valve replacement for a previously implanted heart valve prosthesis, for example, the previously implanted heart valve prosthesis 100. The heart valve prosthesis 300 is configured to reduce coronary artery obstruction, reduce sinus sequestration, and improve the coronary perfusion pathways, as described below. In embodiments herein, the heart valve prosthesis 300 includes a frame 302 supporting a valve assembly 306 and a plurality of gripper pads 400, as described below. In the embodiment shown in FIGS. 3A-3B, the frame 302 is balloon expandable such that the heart valve prosthesis 300 includes a radially compressed or crimped configuration for delivery within a vasculature as shown in FIG. 3A, and a radially expanded configuration for deployment as a valve-in-valve replacement as shown in FIG. 3B.

In embodiments herein, the frame 302 includes an outflow portion 312, an inflow portion 314, and a transition portion 316 bridging, connecting, or otherwise extending between the outflow portion 312 and the inflow portion 314. The frame 302 is a tubular structure that defines a central lumen 304 extending from an inflow end 318 to an outflow end 320. The frame 302 is configured to support the valve assembly 306. As best shown in FIG. 3B, the frame 302 includes a plurality of struts 322 and a plurality of crowns 324, with each crown 324 formed between a pair of opposing struts 322. Each crown 324 is a curved segment, extending between opposing struts 322. The frame 302 includes a plurality of side openings 326 defined by the plurality of struts 323 and the plurality of crowns 324. In embodiments herein, the plurality of side openings 326 may be substantially diamond shaped when the heart valve prosthesis 300 is in the radially expanded configuration. The frame 302 includes a plurality of nodes 328, defined as a region where two crowns 324 meet or connect. In embodiments herein, the frame 302 may be formed by methods such as, but not limited to laser cutting or etching a metal alloy tube comprising, for example, stainless steel or nickel titanium. While shown with a specific number of struts 322 and crowns 324 in FIGS. 3A-3B, this is not meant to be liming, and one skilled in the art will realize that more or fewer struts and crowns and defined side openings are possible. It will be recognized by one skilled in the art that the plurality of side openings 326 may have different sizes, for example, one or more of the side openings 326 located within the outflow portion 312 of the frame may be relatively larger than the side openings 326 of the inflow portion 314 and may be configured to provide access to adjacent coronary arteries for future interventions.

The valve assembly 306 includes an inner skirt 310 and a plurality of valve leaflets 308. The valve assembly 306 is configured to regulate flow therethrough via the plurality of valve leaflets 308. In the embodiments herein, the valve assembly 306 is configured with three valve leaflets 308, and the heart valve prosthesis 300 is configured as a valve-in-valve replacement for a previously implanted aortic heart valve prosthesis. However, the heart valve prosthesis 300 is not required to have three valve leaflets 308, or a tricuspid configuration, and may be configured with two valve leaflets. The plurality of valve leaflets 308 may be coupled at an outflow end to one of a plurality of commissure posts 338 of the frame, and to the inner skirt 310 at an inflow end. The inner skirt 310 may be coupled to the frame 302. The plurality of valve leaflets 308 may be sutured or otherwise securely and sealingly attached along their bases to the inner skirt 310. Adjoining pairs of valve leaflets 308 may be attached to each other at their lateral ends to form commissures 330 with the edges of the valve leaflets 308 forming coaptation edges that meet in an area of coaptation, as would be known to one of ordinary skill in the art. Coupling of the plurality of valve leaflets 308 to the inner skirt 310 and the frame 302, and coupling of the inner skirt 310 to the frame 302 may be by methods such as, but not limited to sutures or a suitable biocompatible adhesive. The plurality of valve leaflets 308 may be formed of various materials, including, but not limited to natural pericardial material, such as tissue from bovine, equine, or porcine, origins, or synthetic materials, such as polytetrafluoroethylene (PTFE), Dacron® polyester, pyrolytic carbon, or other bio compatible materials. The inner skirt 310 may be formed of materials, including, but not limited to materials such a natural or biological material, such as pericardium or other membranous tissue, such as intestinal submucosa, or may be a low, porosity, woven fabric such as polyester, Dacron® fabric, or polytetrafluoroethylene (PTFE).

As noted above, the general description of the heart valve prosthesis 300 is not meant to be limiting. Further, for the embodiments described below, the heart valve prosthesis 300 is generally similar for each embodiment. Therefore, the same reference numbers will be used for the embodiments shown, and differences in the embodiments will be described.

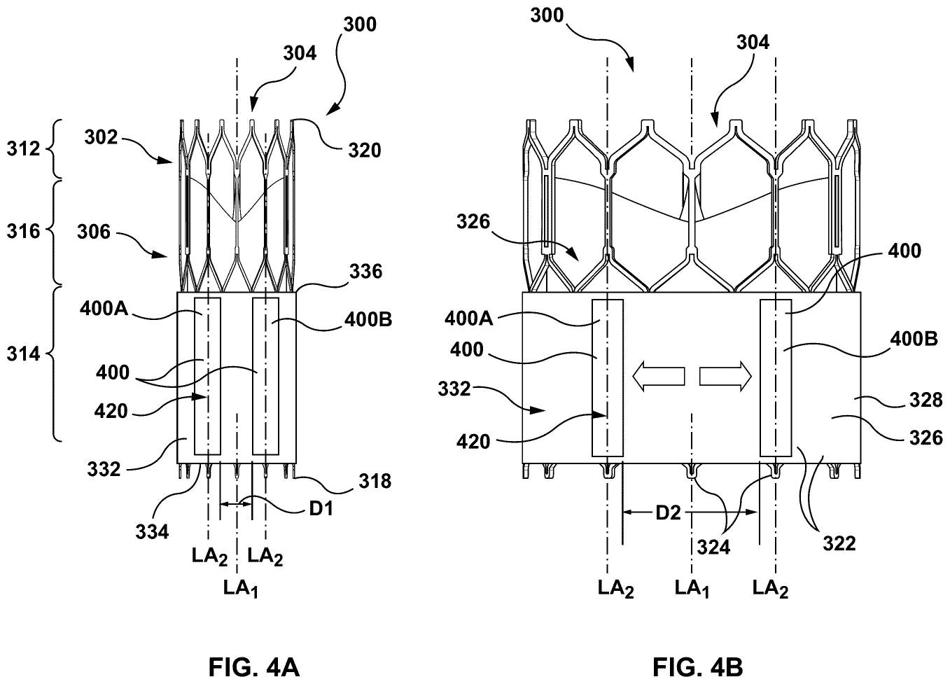

In the embodiments hereof, the heart valve prosthesis 300 further includes the plurality of gripper pads 400 disposed on an outer surface of the heart valve prosthesis. Each gripper pad 400 is configured to engage tissue of a corresponding valve leaflet of a previously implanted heart valve prosthesis, as described below. In embodiments herein, each gripper pad 400 may be a substantially rectangular shape having a proximal end 404, a distal end 406, a first side 408, and a second side 410. However, this is not meant to be limiting, and the gripper pads 400 may be any shape suitable for separating lacerated valve leaflets of the previously implanted heart valve prosthesis. Each gripper pad 400 may further include an inner surface for contacting an outer surface of the frame 302 (FIGS. 3A-3B) or an outer surface of an outer skirt (FIGS. 4A-4B), and an outer surface configured to contact leaflets of a previously implanted heart valve prosthesis, as described below. The outer surfaces of the gripper pads 400 may further include connectors 420, described below, for engagement with the leaflets of the previously implanted heart valve prosthesis. In embodiments herein, each gripper pad 400 is configured to move radially outward with the radial expansion of the heart valve prosthesis 300 when the heart valve prosthesis 300 transitions from the radially compressed configuration to the radially expanded configuration. Due to the radial expansion of the heart valve prosthesis 300, each gripper pad 400 also circumferentially separates from an adjacent gripper pad 400 due to radial expansion of the heart valve prosthesis 300, as described in more detail below.

FIGS. 3A-3B and FIGS. 4A-4B show two embodiments for coupling the gripper pads 400 to the heart valve prosthesis 300. In the embodiment of FIGS. 3A-3B, the gripper pads 400 are attached to the frame 302 of the heart valve prosthesis 300. For example, and not by way of limitation, the gripper pads 400 of the embodiment of FIGS. 3A-3B may be sutured or otherwise coupled to nodes 327 in the inflow portion 314 of the frame 302. However, this is not meant to be limiting, and the gripper pads 400 of the embodiment of FIGS. 3A-3B may also be coupled to the struts 322 of the inflow portion 314 of the frame 302, and/or to the inner skirt 310 of the heart valve prosthesis 300.

Similarly, in the embodiment of FIGS. 4A-4B, the gripper pads 400 are attached to an outer skirt 332 of the heart valve prosthesis 300. The outer skirt 332 is attached to an outer surface of the frame 302 in the inflow portion 314 of the frame 302. In the embodiment shown in FIGS. 4A-4B, the outer skirt 332 includes an inflow end 334 and an outflow end 336. The outer skirt 332 may be coupled to the outer surface of the frame 302 with the inflow end 334 of the outer skirt 332 adjacent the inflow end 318 of the frame 302, and the outflow end 336 of the outer skirt 332 adjacent a distal end of the inflow portion 314 of the frame 302. The outer skirt 332 may be coupled to the frame 302 with methods such as, but not limited to sutures or a suitable biocompatible adhesive. The outer skirt 332 may be formed of materials, including, but not limited to materials such a natural or biological material, such as pericardium or other membranous tissue, such as intestinal submucosa, or may be a low, porosity, woven fabric such as polyester, Dacron® fabric, or polytetrafluoroethylene (PTFE).

In both embodiments the outer surfaces of the gripper pads 400 are facing radially outward from the heart valve prosthesis 300 such that the gripper pads 400 may engage leaflets of a previously implanted heart valve prosthesis, as described below.

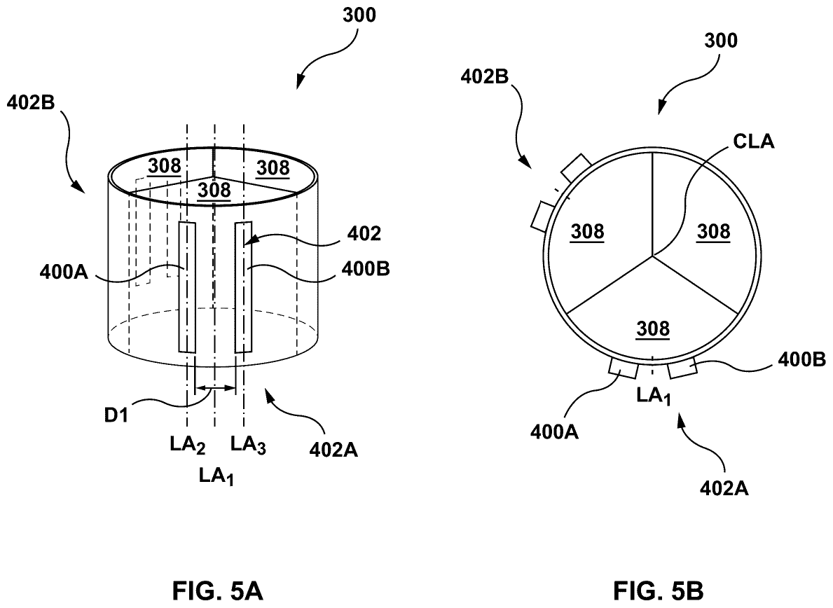

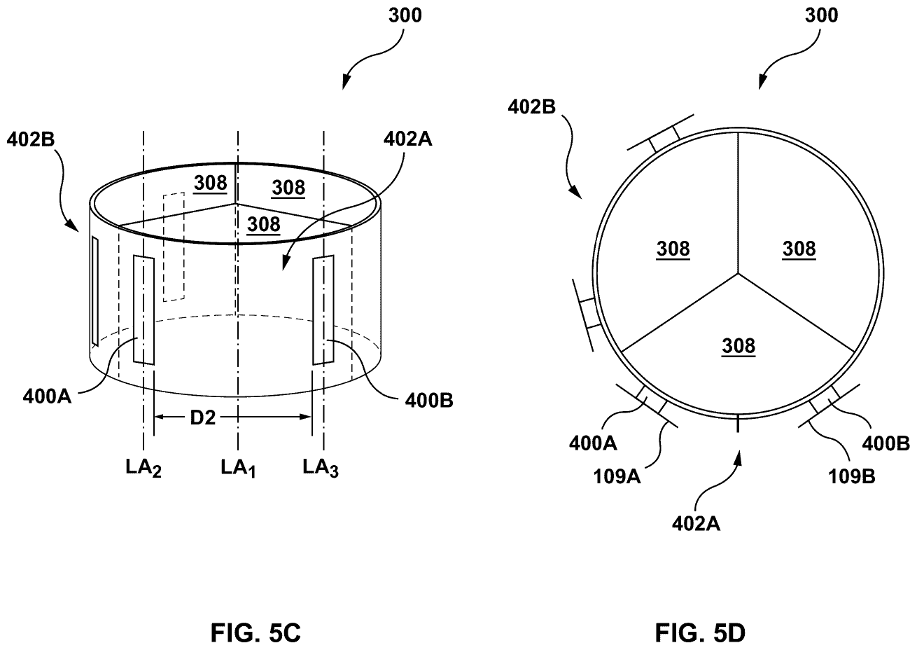

With reference to FIGS. 3A-3B, 4A-4B, and 5A-5D, the grippers pads of the embodiment of FIGS. 3A-3B and 4A-4B are disposed substantially parallel to a central longitudinal axis CLA of the heart valve prosthesis 300, in both the radially compressed configuration and the radially expanded configuration. With reference to FIGS. 5A-5D, which schematically display the embodiments of FIGS. 3A-3B and 4A-4B with the skirts not shown for convenience, The gripper pads 400 may be disposed on the heart valve prosthesis 300 in gripper pad pairs 402. For example, and not by way of limitation, the embodiments of FIGS. 3A-3B and 4A-4B may include two gripper pad pairs 402A, 402B, as shown in FIGS. 5A-5D. Each gripper pad pair 402A, 402B may include two gripper pads 400A, 400A. As shown in FIGS. 5A-5D, each gripper pad pair 402 is associated with one of the leaflets 308 of the heart valve prosthesis 300. In other words, each gripper pad pair 402 is disposed between adjacent the commissure posts 338 of the frame 302 of the heart valve prosthesis 300. In the embodiment shown in FIG. 5A-5D there are two gripper pad pairs 402. In such embodiments, as described in more detail below, the heart valve prosthesis 300 will be aligned with the two sinuses of the native aortic heart valve that include the coronary arteries. However, in other embodiments, the heart valve prosthesis may include three gripper pad pairs 402 such that the heart valve prosthesis 300 need only be aligned with the leaflets 108 of the previously implanted heart valve prosthesis 100.

As noted above, in the embodiments of FIGS. 3A-5D, gripper pads 400 are substantially parallel to a central longitudinal axis CLA of the heart valve prosthesis 300. Further, adjacent gripper pads 400A, 400B of each gripper pad pair 402 are configured to separate as the frame 302 of the heart valve prosthesis 300 radially expands. Therefore, in the radially compressed configuration, as shown in FIGS. 3A, 4A, and 5A-5B, the gripper pads 400A, 400B of the gripper pad pair 402A are disposed on either side of a longitudinal axis LA₁ of the heart valve prosthesis 300 and along second and third longitudinal axes LA₂, LA₃, respectively. The adjacent side edges 410, 408 of the gripper pads 400A, 400B are disposed a first distance D1 from each other (the first distance D1 is an arc length around the circumference of the frame 302). In embodiments, the first distance D₁ may be in the range of about 0 mm (i.e., the side edges abutting or nearly abutting each other) to about 3 mm. Upon expansion of the frame 302 such that the heart valve prosthesis 300 is in the radially expanded configuration, as shown in FIGS. 3B, 4B, and 5C-5D, the gripper pads 400A, 400B of the gripper pad pair 402A expand with the heart valve prosthesis such that the side edges 410, 408 of adjacent gripper pads 400A, 400B are disposed a second distance D2 from each other in the radially expanded configuration. In embodiments, the second distance D2 (also an arch length) may be in the range of about 7 mm to about 18 mm. As described below in the method, the gripper pads 400A, 400B are engaged with a lacerated leaflet of a previously implanted heart valve prosthesis with the heart valve prosthesis 300 in the radially compressed configuration. Therefore, upon expansion of the heart valve prosthesis 300, the gripper pads 400A, 400B radially expand and separate, thereby separating the lacerated leaflet of the previously implanted heart valve prosthesis or native heart valve.

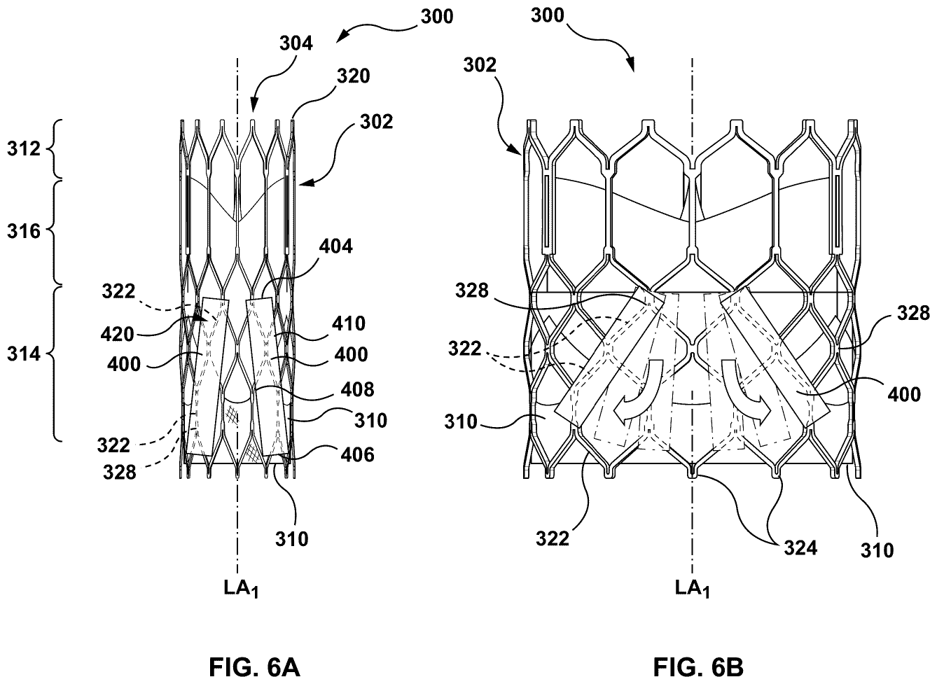

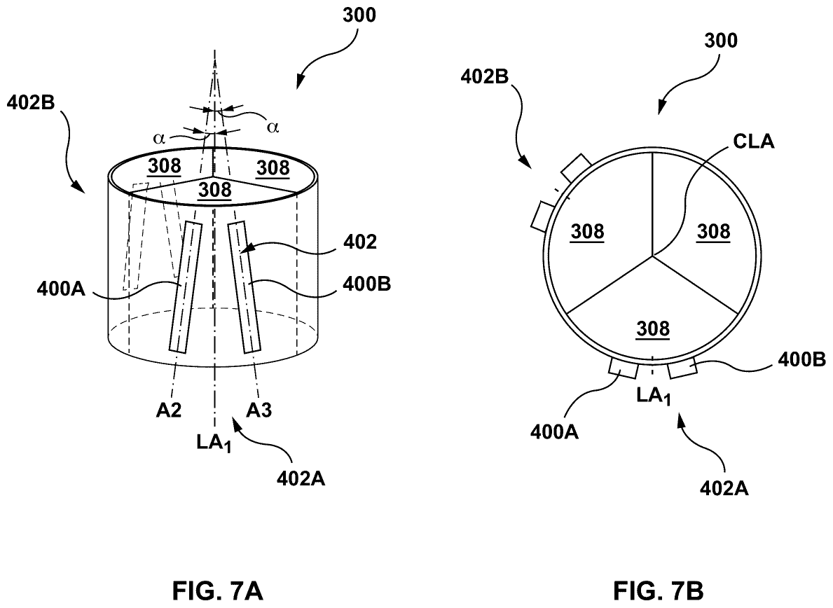

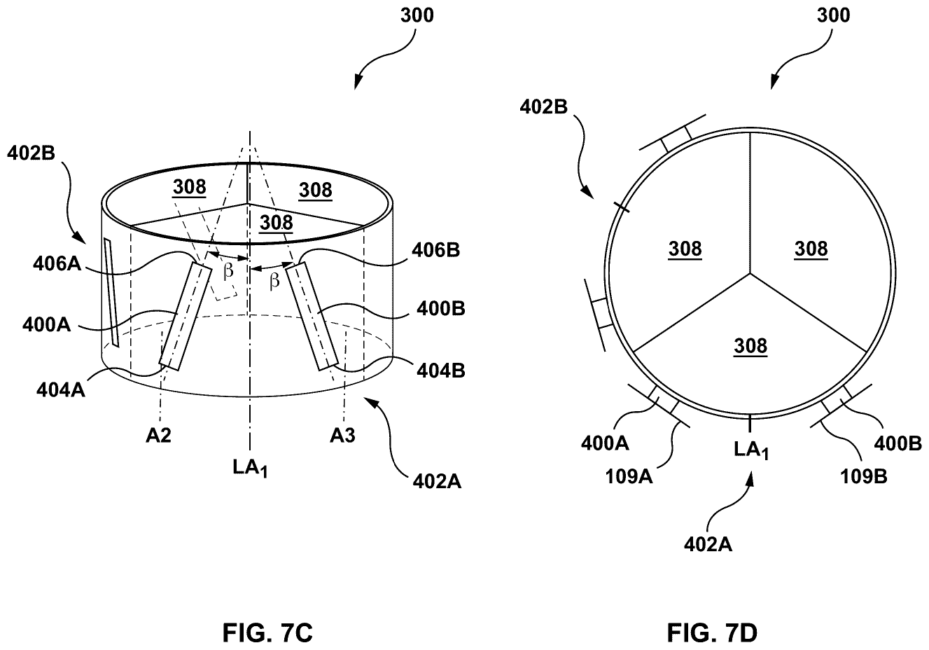

FIGS. 6A-6B and 7A-7D show another embodiment of gripper pads 400 disposed on the heart valve prosthesis 300. The heart valve prosthesis 300 shown in FIGS. 6A-6B and 7A-7D may be as described above with respect to FIGS. 3A-3B. Therefore, the details will not be repeated. Further, as noted above, the heart valve prosthesis 300 is exemplary and other heart valve prostheses may be used. In the embodiment of FIGS. 6A-6B and 7A-7D, the gripper pads 400 are coupled to the struts 322 in the inflow portion of the frame 302 of the heart valve prosthesis. In FIGS. 6A-6B, the frame 302 is shown without an outer skirt coupled thereto. However, in other embodiments, an outer skirt may be coupled to the outer surface of the frame 302. In such an embodiment, the gripper pads 400 may be coupled to the struts 322 through the outer skirt.

As can be seen in FIGS. 6A-6B, with the gripper pads 400 coupled to the frame 302 along the struts 322 of the frame 302, the gripper pads 400 are not disposed parallel to the central longitudinal axis of the heart valve prosthesis. Instead, in the radially compressed configuration shown in FIGS. 6A and 7A, each gripper pad 400A, 400B of a first gripper pad pair 402A is disposed on opposite sides of a first longitudinal axis LA₁ that is parallel to the central longitudinal axis of the heart valve prosthesis 300. The gripper pads 400A, 400B are also disposed along axes A2, A3 which are disposed at an angle α with respect to the first longitudinal axis LA₁. In an embodiment, the angle α is in the range of 0°-45°. The gripper pads 400 of the second gripper pad pair 402B may similarly be disposed at an angle relative to a longitudinal axis disposed therebetween. Further, as noted above, although two gripper pad pairs 402 are shown, more or fewer gripper pad pairs 402 may be utilized depending on the application. For example, and not by way of limitation, three gripper pad pairs may be utilized, corresponding to three native leaflets of a native aortic valve.

Similar to the embodiments of FIGS. 3A-3B and 4A-4B, when the heart valve prosthesis 300 is radially expanded from the radially compressed configuration to the radially expanded configuration, the gripper pads 400 move with the frame 302 of the heart valve prosthesis 300. Thus, in the embodiment of FIGS. 6A-6B, because the gripper pads 400 are coupled to the struts 322 of the frame 302 and the struts 322 rotate when expanded, the gripper pads 400 also rotate. In particular, as shown in FIGS. 6B and 7C, the axes A2 and A3 rotate such that an angle β of each axis A2, A3 relative to the first longitudinal axis is larger than the angle α. In an example, the angle β may be in the range of 0°-90°. Therefore, upon expansion of the heart valve prosthesis 300, the gripper pads 400A, 400B of FIGS. 6A-6B and 7A-7D radially expand and separate, thereby separating the lacerated leaflet of the previously implanted heart valve prosthesis or native heart valve.

While the embodiments described show the gripper pads 400 coupled to particular portions of the heart valve prosthesis 300, having a particular shape and orientation, and the heart valve prosthesis 300 having particular parts in a particular arrangement, this is not meant to be limiting, and the gripper pads may be coupled to other portions of the heart valve prosthesis 300, have different shapes and orientations, and the heart valve prosthesis may have different, more, or fewer features in keeping with the spirit and scope of this disclosure. The gripper pads 400 are oriented outwardly such that the gripper pads 400 are configured to engage the leaflets of a previously implanted heart valve prosthesis, and the gripper pads 400 are configured separate as the heart valve prosthesis 300 is radially expanded such that the gripper pads 400 separate the lacerated leaflets of the previously implanted heart valve prosthesis or native heart valve.

The gripper pads 400 may be made of a material that interacts with tissue (i.e., tissue of the leaflets 108 of the previously implanted heart valve prosthesis 100) in a hook-and-loop style. For example, and not by way of limitation, the material may be Medtronic's ProGrip™ Self-Fixating Mesh (Monofilament Polyethylene Terephthalate (PET) textile material with Monofilament Polylactic Acid (PLA) microgrip material. In other embodiments, the gripper pads may be made of Polypropylene, Nylon, Nitinol, Polylactic-co-glycolic acid (PLGA), Polyglycolic acid (PGA), Stainless steel, cobalt-chromium.

As explained above, the gripper pads 400 include a mesh material having hook-and-loop style hooks or barbs that engage the tissue of the leaflets 108 of the previously implanted heart valve prosthesis 100. The material requires low force to engage the leaflets 108 axially, but requires high force to disengage in shear. Therefore, the material will not disengage from the leaflets 108 of the previously implanted heart valve prosthesis 100 when the heart valve prosthesis 300 is radially expanding and the gripper pads 400 are separating the leaflets 108 in shear. Further, flexible hooks or barbs enable high packing density/lower profile of the heart valve prosthesis 300 with the gripper pads 400.

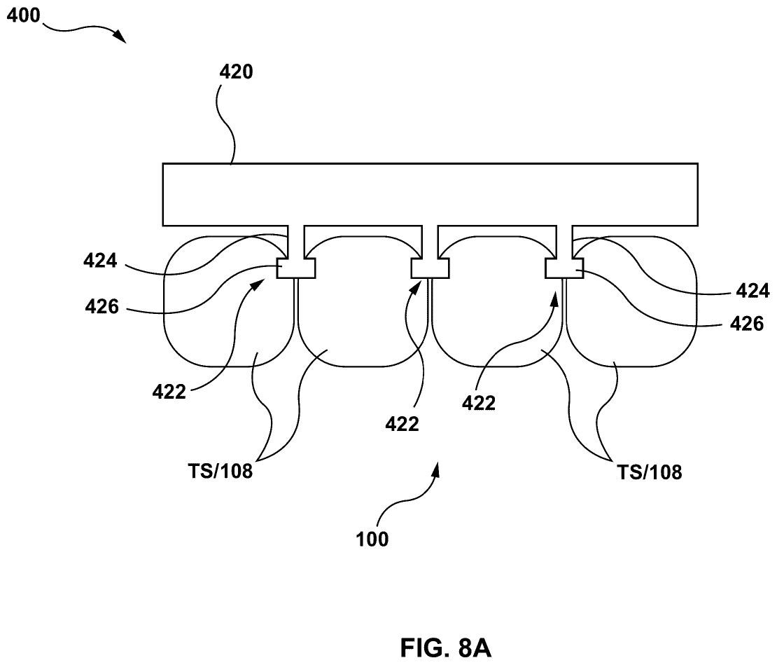

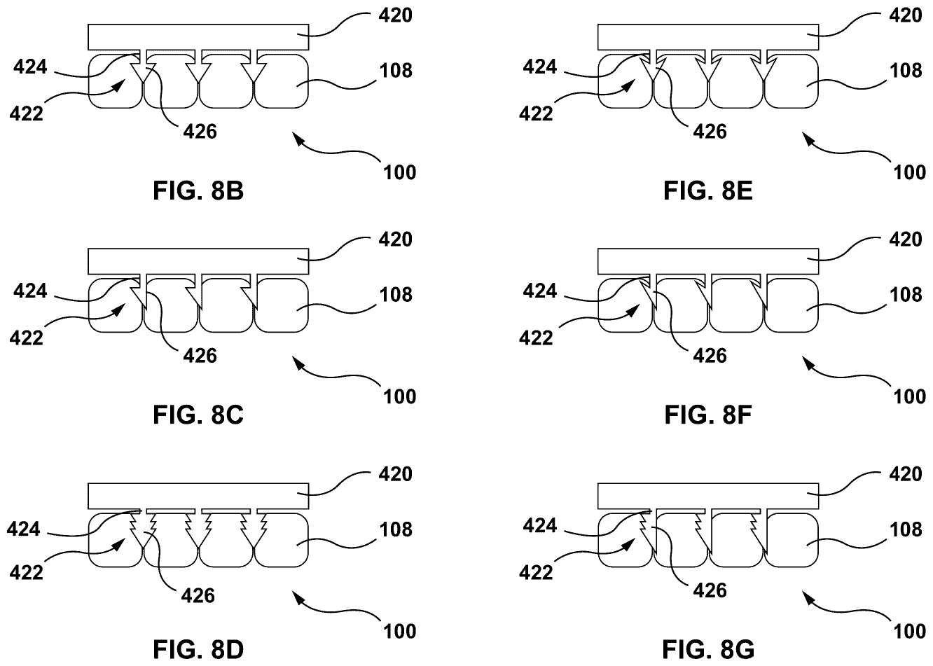

FIGS. 8A-8G shows various embodiments of gripper pads 400 with different types of barbs. In particular, the gripper pads 400 include a base 420 and hooks 422 extending from the base 420. As noted above, the base 420 may be the textile material described above, and the hooks 422 may be the microgrip material described above. The hooks 422 may include a post or shank 424 extending from the base 420 and a barb 426 disposed on the post 424 opposite the base 420. FIGS. 8A-8G show various embodiments of the barbs 426, such as transverse barbs, arrow type barbs, half-arrow barbs, multiple arrow barbs, and multiple half-arrow barbs. However, this is not meant to be limiting, and other barbs may be utilized. Further, it is noted that the gripper pads 400 are unlikely to stick to an introducer sheath or capsule of a delivery system as the gripper pads are configured to engage with tissue fibers only, and therefore are unlikely to engage with a hard polymer or metal interface on an inside diameter of an introducer sheath or capsule of a delivery system. In embodiments hereof, the gripper pads 400 may be optimally designed with a free end of each barb 426 of each hook 422 pointing in a specific direction, with the plurality of hooks 422 laying down flat for delivery, or with each gripper pad 400 disposed in areas of low packing density to minimize the profile of the heart valve prosthesis 300 in the radially compressed configuration.

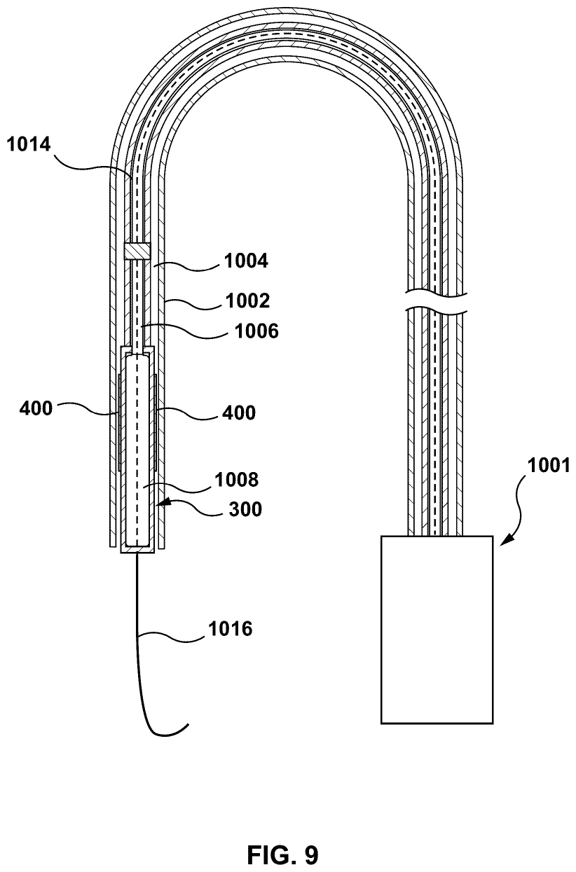

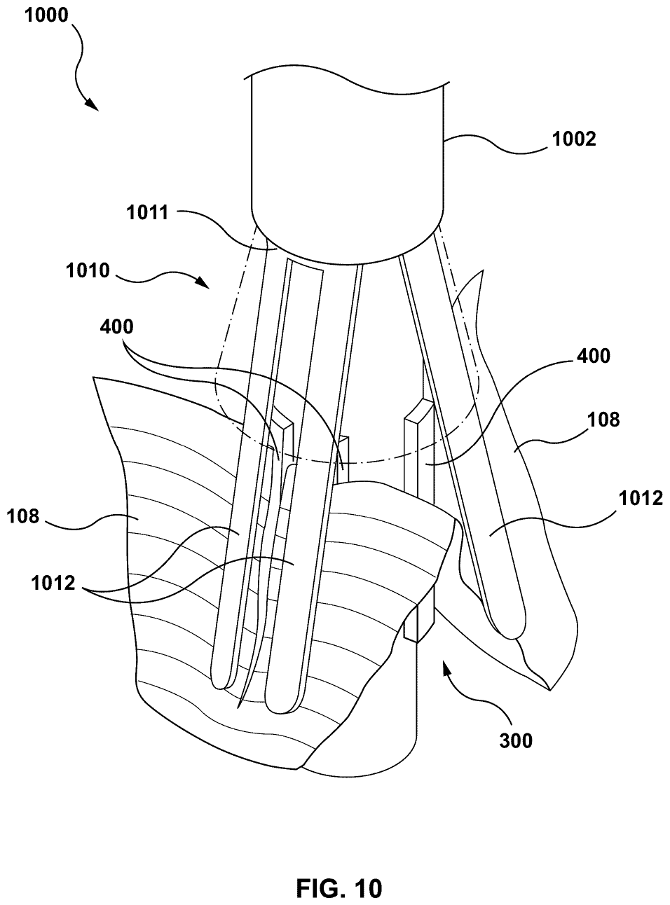

FIGS. 9-12 show embodiments of a distal portion of a delivery system 1000 suitable for percutaneously delivering and implanting the heart valve prosthesis 300, in accordance with embodiments herein. The delivery system 1000 may assume different forms and construction based upon application needs as described in greater detail in U.S. Pat. No. 8,579,963, which is incorporated in its entirety by reference herein. The delivery system 1000 is configured to be used for transcatheter valve-in-valve replacement. It is understood that other embodiments of the delivery system 1000 are possible. The delivery system 1000 is merely an exemplary embodiment of a transcatheter delivery system and modifications can be made to the various embodiments describe herein without departing from the spirit and scope of the present disclosure. Therefore, the following detailed description is not meant to be limiting. Further, the systems and functions described below can be implemented in many different embodiments of hardware. Any actual hardware described is not meant to be limiting. The operation and behavior of the systems and methods presented are described with the understanding that modifications and variations of the embodiments are possible given the level of detail presented.

In general, the delivery system 1000, and other delivery system embodiments in accordance with the principles of the present disclosure, provide features capable of retaining and delivering a replacement heart valve prosthesis, such as the heart valve prosthesis 300, to the site of a previously implanted heart valve prosthesis, such as the previously implanted heart valve prosthesis 100, along with mechanisms for adjusting the position of the replacement heart valve prosthesis 300 within the previously implanted heart valve prosthesis 100. Further, the delivery system 100 includes mechanisms to selectively engage the valve leaflets 108 of the previously implanted heart valve prosthesis 100 with the gripper pads 400 of the replacement heart valve prosthesis 300.

Referring to FIG. 9, the delivery system 1000 includes a handle 1001, an outer shaft 1002 defining an outer shaft lumen 1004 operably coupled to the handle 1001, and an inner shaft 1006. The inner shaft 1006 is disposed within the outer shaft lumen 1004 of the outer shaft 1002. The inner shaft 1006 includes an inner shaft lumen 1014 sized to receive an auxiliary component such as a guidewire 1016. The inner shaft 1006 includes an expandable balloon 1008 disposed at a distal end thereof. The expandable balloon 1008 is configured to expand the heart valve prosthesis 300 from the radially compressed configuration to the radially expanded configuration within the previously implanted heart valve prosthesis 100. The expandable balloon 1008 is configured to press the plurality of gripper pads 400 of the heart valve prosthesis 300 into the corresponding lacerated valve leaflets 108 of the previously implanted heart valve prosthesis 100, as illustrated in FIG. 10. The delivery system 1000 includes the heart valve prosthesis 300 disposed or loaded on an outer surface of the expandable balloon 1008.

The delivery system 1000 includes a leaflet clip mechanism 1010 disposed between the outer shaft 1002 and the inner shaft 1006 (see FIG. 9) and operably coupled to the handle 1001 (e.g. coupled to an actuator (not shown) of the handle 1001). During delivery, the leaflet clip mechanism 1010 is configured to selectively engage the corresponding lacerated valve leaflets 108 of the previously implanted heart valve prosthesis 100 and press the adjacent lacerated valve leaflets 108 into the corresponding plurality of gripper pads 400 of the heart valve prosthesis 300. Stated another way, the leaflet clip mechanism 1010 ensures sufficient frictional force is achieved to engage the gripper pads 400 of the heart valve prosthesis 300 with the corresponding valve leaflets 108 of the previously implanted heart valve prosthesis 100.

In the embodiment of FIG. 10, the leaflet clip mechanism 1010 includes a leaflet clip shaft 1011 and a plurality of fingers 1012 extending distally from the leaflet clip shaft 1011. Each finger 1012 may be a generally elongated rectangular shape with a proximal end coupled to the leaflet clip shaft and a free distal end. Each finger 1012 is radially aligned with a corresponding gripper pad 400 of the heart valve prosthesis 300. When selectively activated by a user, each finger 1012 is configured to selectively open radially outward when the fingers 1012 are extended from the outer shaft 1002 to extend over the corresponding lacerated leaflet 108, engage with the outer surface of the lacerated leaflet 108, and impart an inward radial force on the corresponding lacerated valve leaflet 108 of the previously implanted heart valve prosthesis 100. More specifically, upon user activation, the fingers 1012 press the corresponding lacerated portions of the lacerated valve leaflets 108 of the previously implanted heart valve prosthesis 100 into the corresponding gripper pads 400 of the heart valve prosthesis 300, to thereby engage or couple each lacerated valve leaflet 108 with the corresponding gripper pad 400. The plurality of fingers 1012 may be formed of various materials including, but not limited to shape memory materials, such as nickel titanium (Nitinol), Stainless Steel, cobalt-chromium, or other suitable materials.

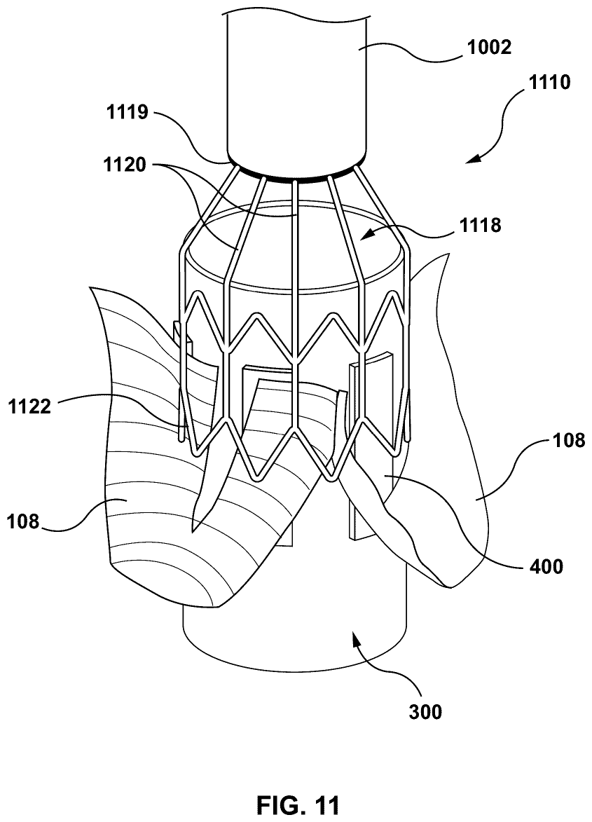

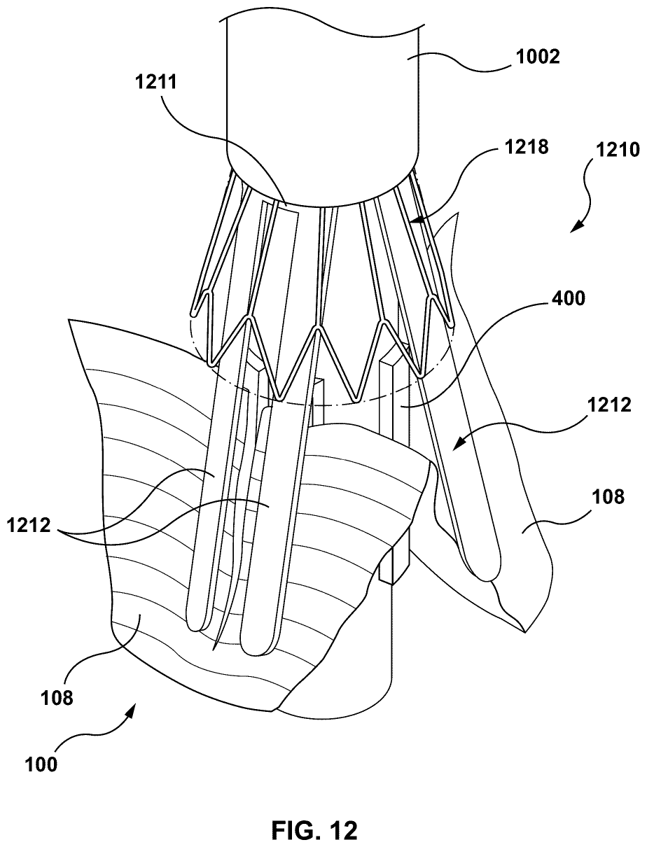

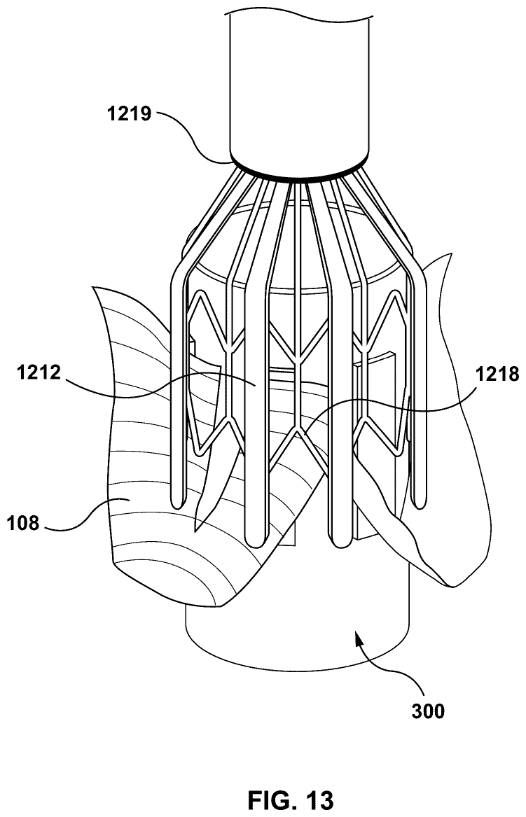

In an embodiment shown in FIG. 11, a leaflet clip mechanism 1110 includes a shaft 1119 and a tension loop 1118 extending distally from the shaft 1119. The tension loop 1118 may be a mesh or web structure formed as a with a plurality of longitudinal members 1120 and at least one circumferential member 1122. The tension loop 1118 is configured to selectively open radially outward when the fingers tension loop is extended from the outer shaft 1002 to extend over the corresponding lacerated leaflet 108, engage with the outer surface of the lacerated leaflet 108, and impart an inward radial force on the corresponding lacerated valve leaflet 108 of the previously implanted heart valve prosthesis 100. The tension loop 1118 may be formed of materials, for example, and not by way of limitation, wire mesh, polymer sutures, or nickel titanium (Nitinol). In an embodiment, the plurality of longitudinal members 1120 of the tension loop 1118 may be stainless steel or nickel titanium (Nitinol) and configured to operate in tension to impart the inward radial force. While shown in FIG. 12 with a specific number of longitudinal members 1120 and circumferential members 122, this is not meant to be limiting, more or fewer longitudinal members 1120 and/or circumferential members 1122 may be utilized.

FIGS. 12-13 illustrate a leaflet clip mechanism 1210 according to embodiments herein. In the embodiment FIGS. 12-13, the leaflet clip mechanism 1210 includes both a plurality of fingers 1212 such as the fingers 1012 of FIG. 10, and a tension loop 1218 such as the tension loop 1118 of FIG. 11. In particular, the leaflet clip mechanism 1210 includes a first shaft 1211 with the plurality of fingers 1212 extending distally from a distal end of the first shaft 1211, as described above with respect to FIGS. 9-10. The leaflet clip mechanism 1210 further includes a second shaft 1219 with the tension loop 1218 extending from a distal end thereof, as explained above with respect to claim 11. The second shaft 1219 is disposed over or radially outside of the first shaft such that the tension loop 1218 is disposed over or radially outside of the fingers 1212. is disposed on an outer surface of the plurality of leaflet clips 1312. The first shaft 1211 and the second shaft 1219 extend proximally to the handle 1001 of the delivery system 1000 (e.g., the shafts may be coupled to actuators to move the first and second shafts 1211, 1219, respectively, and thereby move the finger 1212 and the tension loop 1218, respectively. The plurality of fingers 1212 may be as described with respect to the fingers 1012 (FIG. 10) and the tension loop 1218 may be as described with respect to the tension loop 1118 (FIG. 11). In the embodiment of FIGS. 12-13, the first shaft 1210 is extended distally out of the outer shaft 1002 such that the fingers 1212 open radially outward. The first shaft 1210 is moved further distally such that the fingers 1212 are extended over the corresponding lacerated leaflets 108 and engage with the outer surface of the corresponding lacerated leaflets 108, as shown in FIG. 12. The second shaft 1219 is then advanced such that the tension loop 1218 extends over the fingers 1212. The tension loop 1218 is biased radially inward such that the tension loop 1218 imparts an inward radial force on the fingers 1212 and hence, the corresponding lacerated valve leaflets 108 of the previously implanted heart valve prosthesis 100. In other words, the tension loop 1218 imparts a radially inward force on the fingers 1212, which impart a radially inward force on the lacerated leaflets 108, thereby pressing the corresponding lacerated portions of the lacerated valve leaflets 108 of the previously implanted heart valve prosthesis 100 into the corresponding gripper pads 400 of the heart valve prosthesis 300, to thereby engage or couple each lacerated valve leaflet 108 with the corresponding gripper pad 400, as shown in FIG. 13. The use of both the fingers 1212 and tension loop 1218 may be preferable such that the fingers 1212 may be biased radially outward to ensure the fingers 1212 extend over the leaflets 108, and the tension loop 1218 may be biased radially inward to impart the radially inward force on the fingers 1212, and hence the leaflets 108.

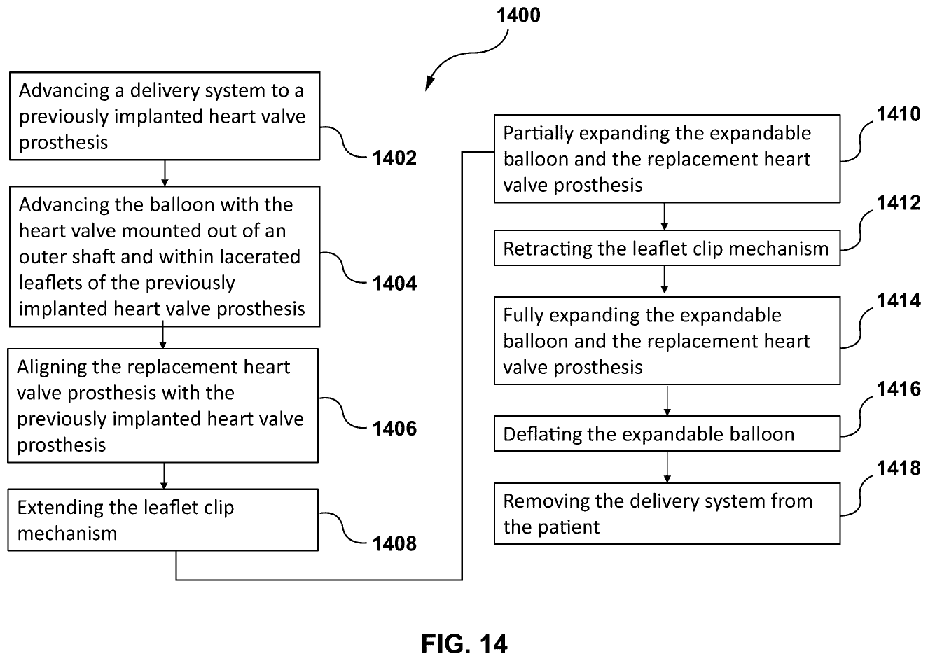

FIGS. 14-24 illustrate a method of delivering and deploying a heart valve prosthesis with a plurality of gripper pads at the site of a previously implanted heart valve prosthesis in accordance with embodiments herein. While described herein with the exemplary heart valve prosthesis 100 as the previously implanted heart valve prosthesis, the heart valve prosthesis 300 as the replacement heart valve prosthesis, and the leaflet clip mechanism 1218 of FIGS. 12-13, this is not meant to be limiting, and other suitable heart valve prostheses and/or embodiments of delivery systems according to embodiments hereof may be utilized. FIG. 14 shows a flow chart with an overview for implanting the heart valve prosthesis 300 within the previously implanted heart valve prosthesis 100 using the delivery system 1000. Those skilled in the art will recognize that FIGS. 14-24 illustrate one example of a method of implanting a previously implanted heart valve prosthesis with a heart valve prosthesis having gripper pads, and that steps of the described method may be removed and/or additional steps may be added. Additionally, the steps described need not be performed in the order described. Further, although the method is described with respect to a valve-in-valve replacement at a native aortic valve, this is not meant to be limiting, and the method may be performed at other native heart valves.

In preparation for the method 1400 described herein, the valve leaflets of the previously implanted heart valve prosthesis are lacerated as described above.

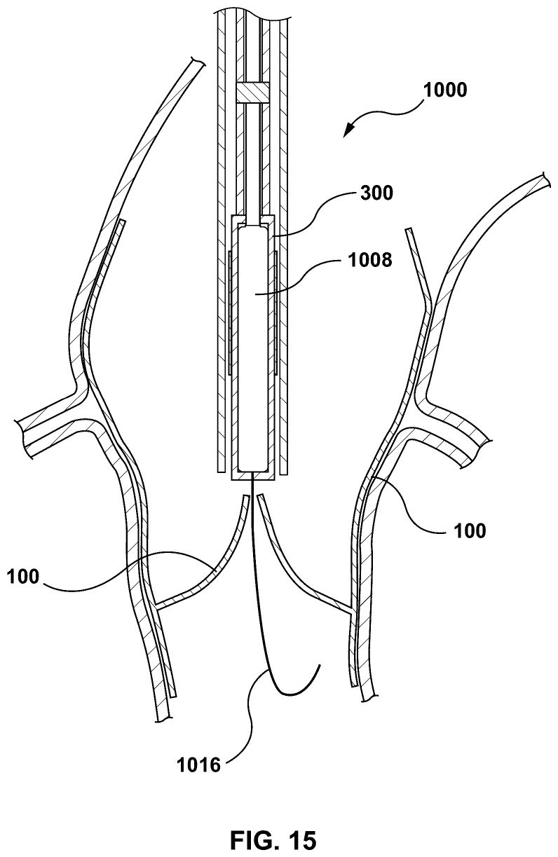

In a step 1402 of the method 1400, the delivery system 1000 is distally advanced, i.e., away from the clinician, through an aorta, and into the previously implanted heart valve prosthesis 100, as illustrated in FIG. 15. The delivery system 1000 includes the heart valve prosthesis 300 in a radially compressed configuration on an outer surface of the expandable balloon 1008, as described previously. The delivery system 1000 and/or the heart valve prosthesis 300 may also include, for example, radiopaque markers such that the clinician may properly align the location of the heart valve prosthesis 300 within the previously implanted heart valve prosthesis 100. In greater detail, the guidewire 1016 is advanced to the site of the previously implanted heart valve prosthesis 100 with the delivery system 1000 disposed over the guidewire 1016. The guidewire 1016 and the delivery system 1000 may be introduced through an opening or arteriotomy through the wall of a femoral artery in the groin region of the patient by known methods, such as, but not limited to, the Seldinger technique. The guidewire 1016 and the delivery system 1000 are advanced into the descending or abdominal aorta, over the aortic arch, the ascending aorta, the aortic sinus, and into the previously implanted (aortic) heart valve prosthesis 100. Further, as shown in FIG. 15, the guidewire 1016 is advanced past the leaflets 108 of the previously implanted heart valve prosthesis 100.

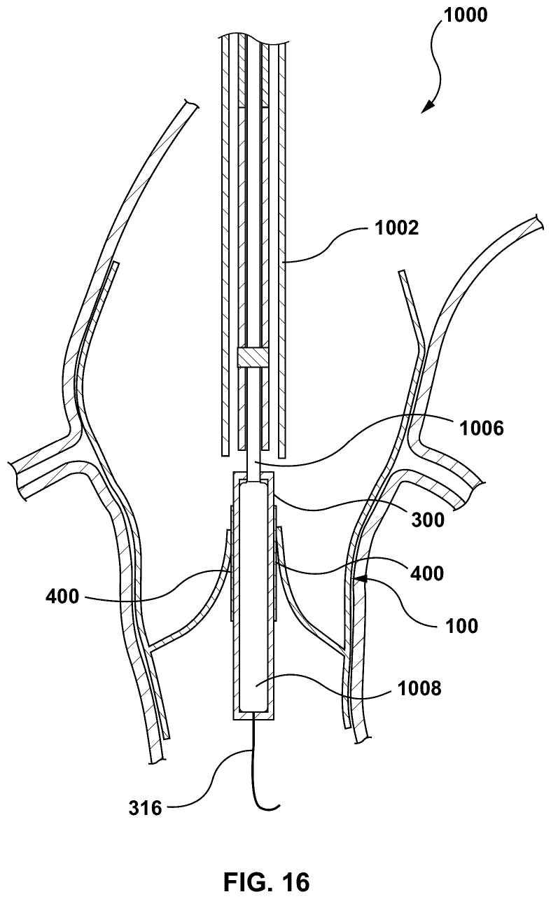

In a step 1404 of the method 1400, with the delivery system 1000 located within the previously implanted heart valve prosthesis 100, the inner shaft 1006 is advanced distally, as shown in FIG. 16. As explained above, the balloon 1008 is coupled to the inner shaft 1006 and the heart valve prosthesis 300 is mounted on the balloon 1008. Therefore, the balloon 1008 and the heart valve prosthesis 300 are advanced distal of the outer shaft 1002 with the inner shaft 1006. The inner shaft 1006, balloon 1008, and heart valve prosthesis 300 are located within the lacerated leaflets 108 of the previously implanted heart valve prosthesis 100, as shown in FIG. 16.

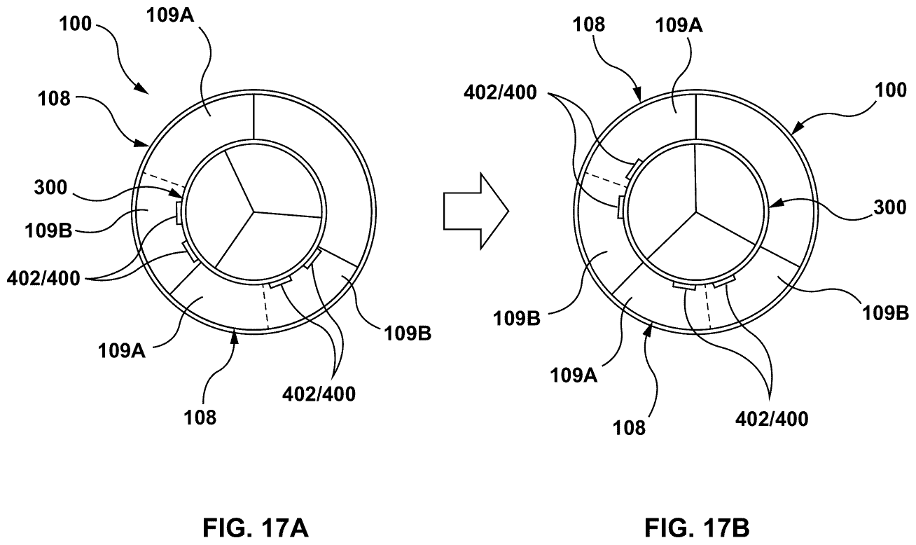

In a step 1406 of the method 1400, as illustrated in FIGS. 17A-17B, the heart valve prosthesis 300 is aligned with the previously implanted heart valve prosthesis 100. More precisely, the heart valve prosthesis 300 is aligned longitudinally with the previously implanted heart valve prosthesis 100 and is aligned rotationally such that each of the plurality of gripper pads 400 of the heart valve prosthesis 300 is aligned with an adjacent and corresponding leaflet portion 109A, 109B of the corresponding lacerated leaflet 108 of the previously implanted heart valve prosthesis 100. The rotational alignment also ensures correct commissure position upon deployment (i.e., the commissures 330 of the valve leaflets 308 of the heart valve prosthesis 300 are aligned with the commissures of the leaflets 108 of the previously implanted heart valve prosthesis 100. As shown in FIG. 17A, the commissures 308 of the valve leaflets 308 of the heart valve prosthesis 300 are not aligned with the commissures of the leaflets 108 of the previously implanted heart valve prosthesis 100. Further, due to this misalignment, the plurality of gripper pads 400 of the heart valve prosthesis 300 are not aligned with the corresponding leaflet portions 109A, 109B of corresponding lacerated valve leaflets 108. Further, in embodiments with two gripper pad pairs 402 (i.e., not three gripper pad pairs) and three valve leaflets 108 of the previously implanted heart valve prosthesis 100, proper rotational alignment may also requires that the two gripper pad pairs 402 are aligned with the two leaflets 108 of the previously implanted heart valve prosthesis 100 in the native sinuses that includes coronary ostia for the coronary arteries. FIG. 17B shows proper rotational alignment as the commissures are aligned and the gripper pads 400 are aligned. Rotation of the heart valve prosthesis 300 may be enabled by a steerable outer shaft 1005 and a torqueable inner shaft 1006. Further, markers on the heart valve prosthesis 300 and/or the delivery system 1000 may be used to ensure proper rotational alignment. The heart valve prosthesis 300 may be properly longitudinally and rotationally aligned using imaging markers. For example, and not by way of limitation, imaging markers and techniques described in U.S. Patent Publication Nos. 2022/0061985; 2022/0061985; and 2022/0175524, each assigned to Medtronic, Inc. and incorporated by reference herein in their entirety, may be used.

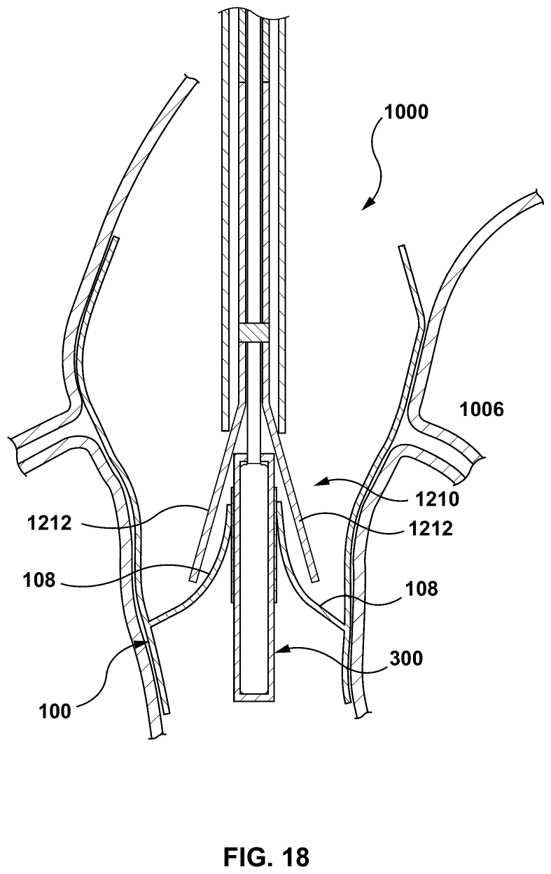

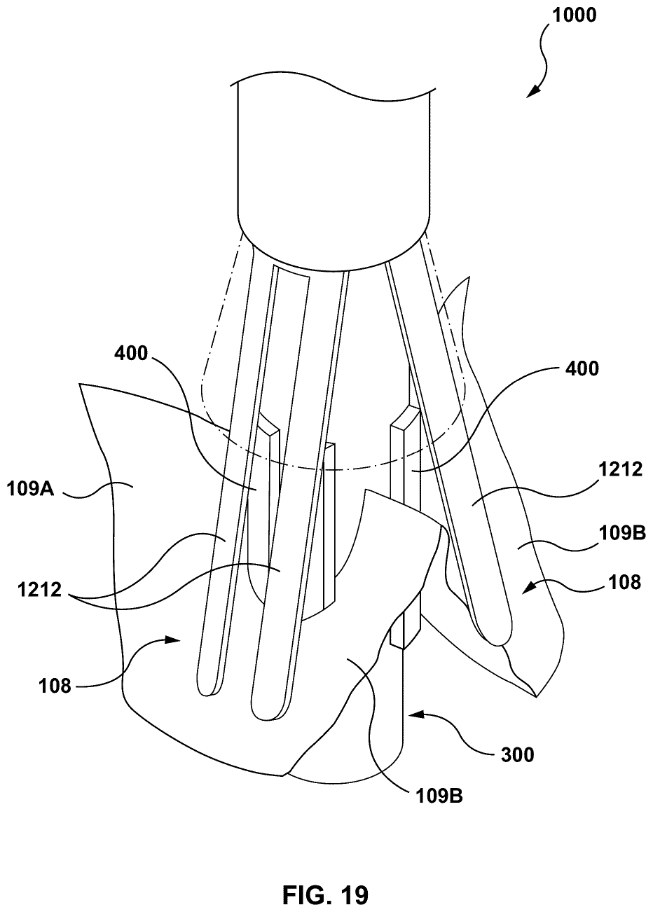

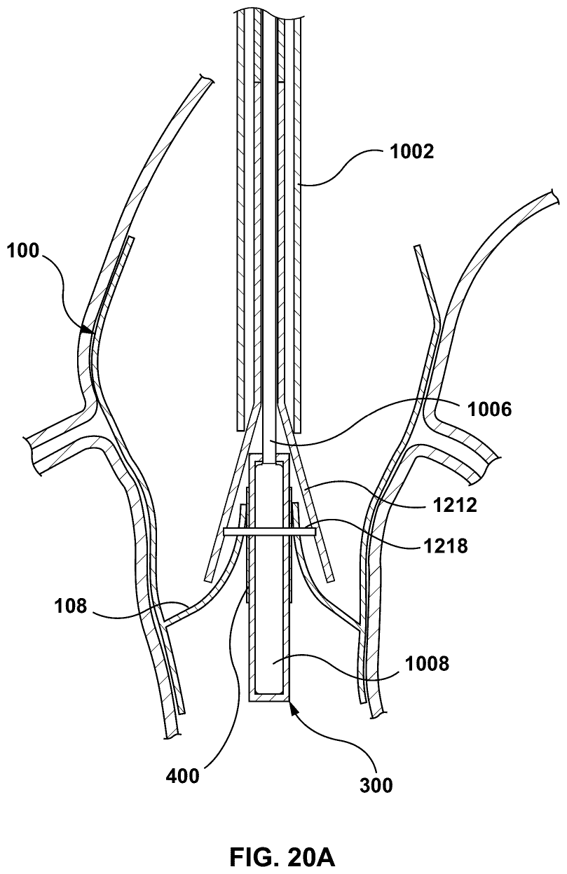

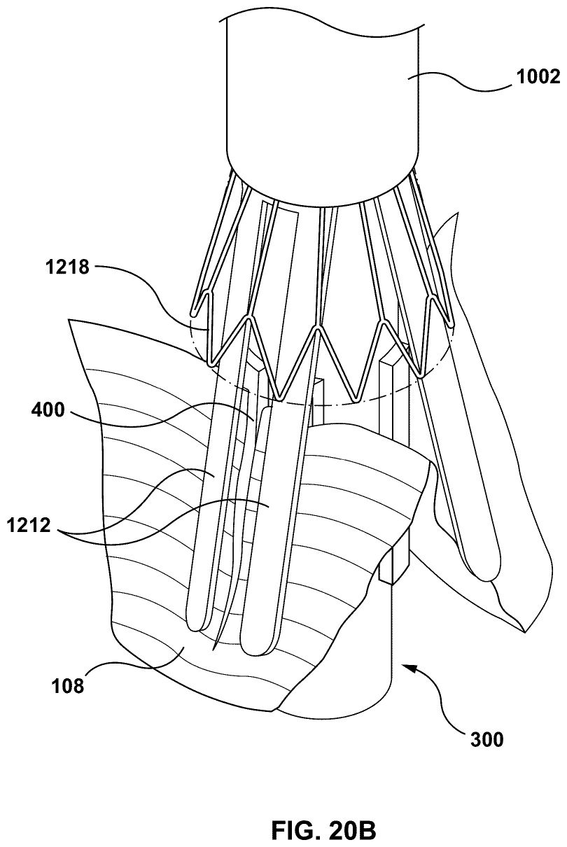

In a step 1408 of the method 1400 the leaflet clip mechanism 1210 of the delivery system 1000 is distally extended. In particular, in the embodiment of the leaflet clip mechanism 1210, the first shaft 1211 is extended distally such that the fingers 1212 extend over the valve leaflets 108 of the previously implanted heart valve prosthesis 100, as shown in FIGS. 18 and 19. With the fingers 1212 in place, the second shaft 1219 is advanced distally such that the tension loop 1218 extends over the fingers 1212, as shown in FIGS. 20A and 20B. As described above, the tension loop 1218 provides a radially inward force pushing the fingers 1212 radially inward.

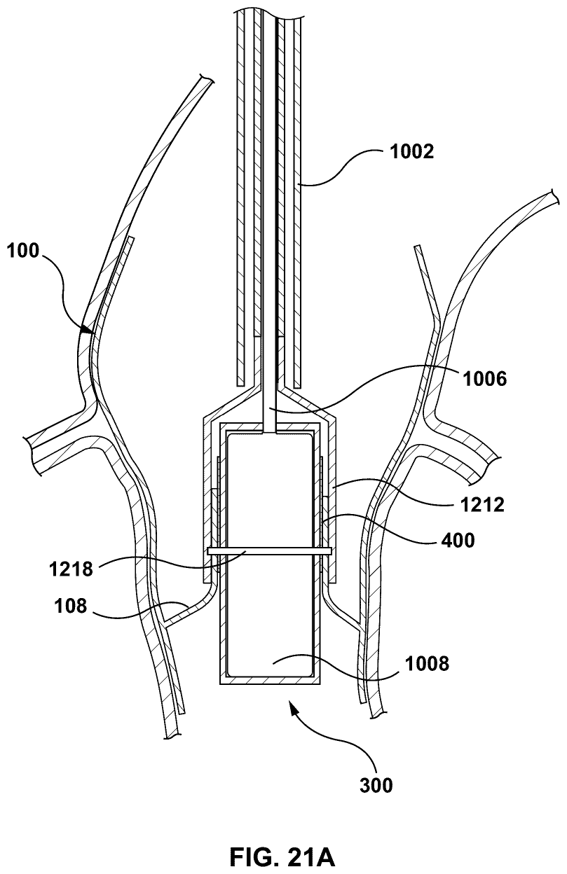

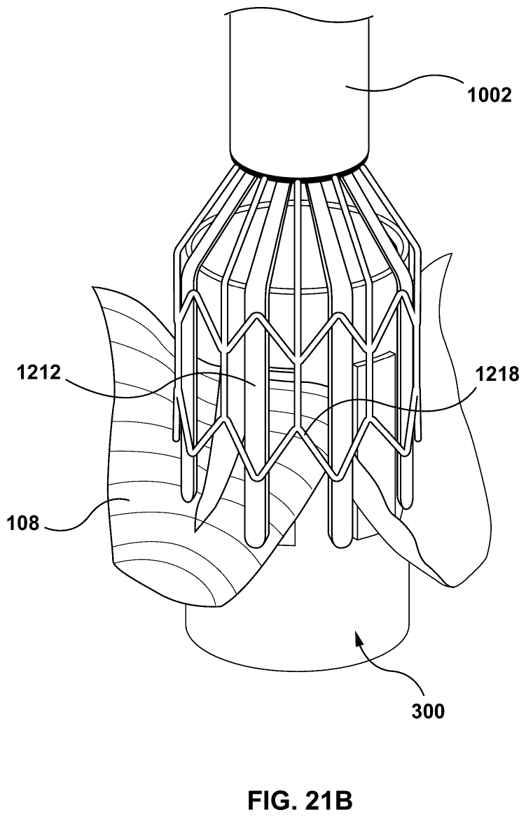

In a step 1410 of the method 1400, as illustrated in FIGS. 21A and 21B, the expandable balloon 1008 of the delivery system 1000 is partially expanded such that the heart valve prosthesis 300 is partially expanded. The expandable balloon 1008 imparts an outward radial force on the heart valve prosthesis 300 and the plurality of gripper pads 400 disposed on the outer surface of the heart valve prosthesis 300. The radially outward force of the expandable balloon 1008 expanding the heart valve prosthesis 300 and the gripper pads 400 thereof from within the valve leaflets 108 of the previously implanted heart valve prosthesis 100 acts against the radially inward force of the leaflet clip mechanism 1210 against an outer surface of the valve leaflets 108 of the previously implanted heart valve prosthesis 100 to engage the plurality of gripper pads 400 of the heart valve prosthesis 300 with the corresponding lacerated leaflet portions 109 of the corresponding valve leaflets 108 of the previously implanted heart valve prosthesis 100.

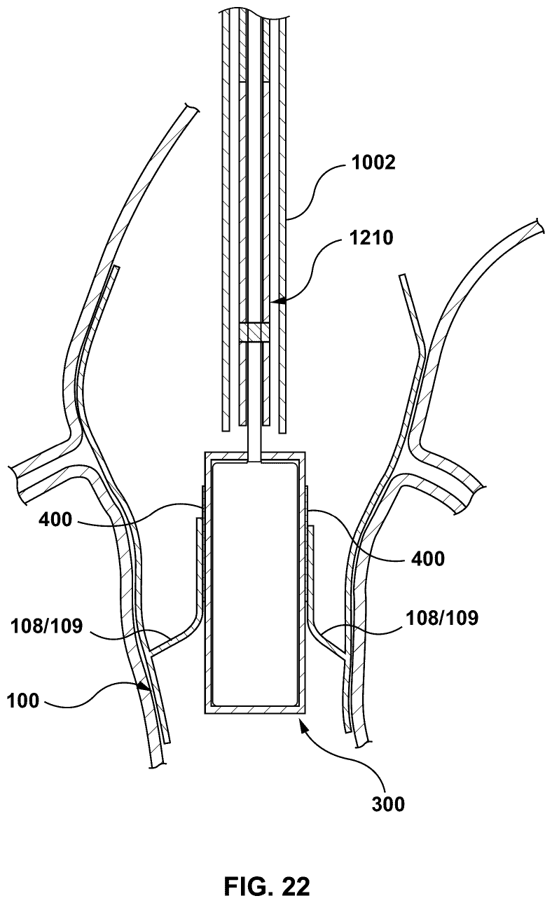

In a step 1412 of the method 1400, with each lacerated leaflet portion 109 of each valve leaflet 108 engaged with a corresponding gripper pad 400 of the heart valve prosthesis 300, the leaflet clip mechanism 1210 may be retracted proximally, i.e., towards the clinician and into the outer shaft 1002 of the delivery system 1000, as shown in FIG. 22. The leaflet clip mechanism 1210 may be retracted by retracting the first shaft 1211 and the second shaft 1219.

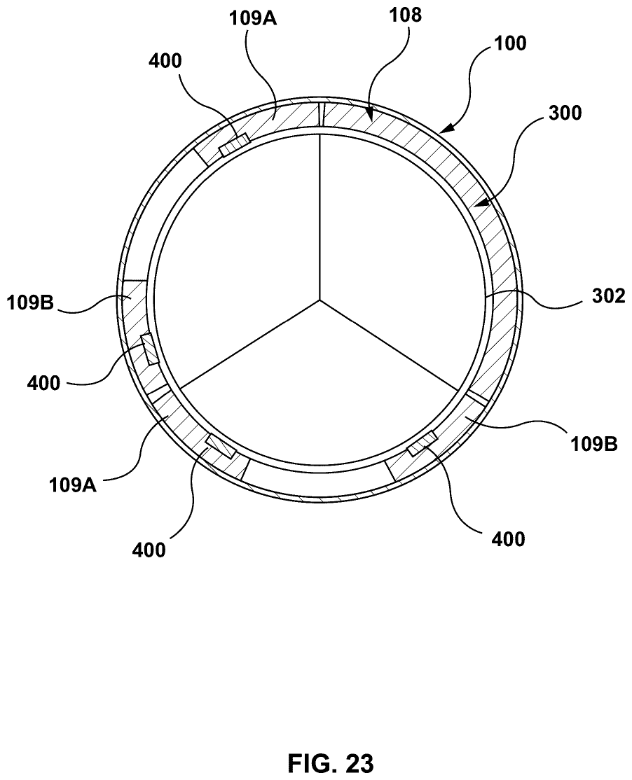

In a step 1414 of the method 1400, the expandable balloon 1008 is fully expanded such that the heart valve prosthesis 300 transitions to the radially expanded configuration and engages with the previously implanted heart valve prosthesis 100, as shown in FIG. 23. As the heart valve prosthesis 300 radially expands to the radially expanded configuration, the gripper pads 400 of each gripper pad pair 402 separate (and/or pivot) away from each other, thereby also separating the lacerated leaflet portions 109A, 109B coupled thereto away from each other, as shown in FIG. 23.

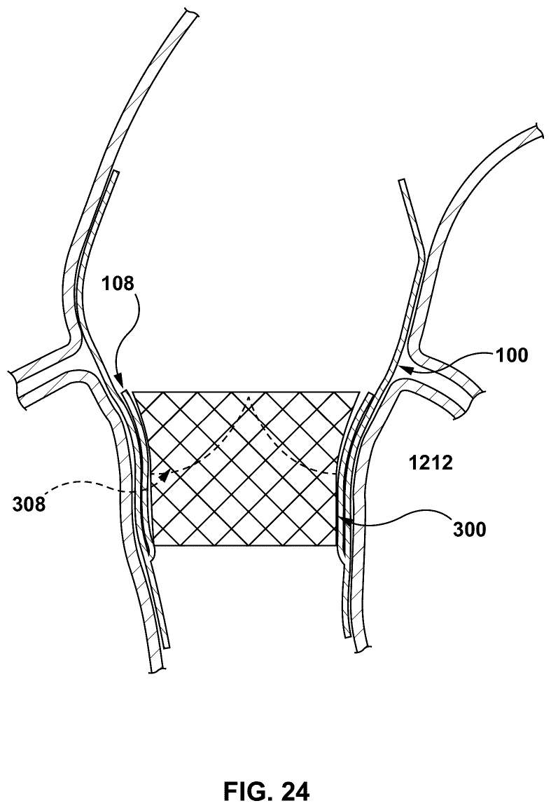

In a step 1416 of the method 1400, with the heart valve prosthesis 300 fully deployed in the radially expanded configuration, the expandable balloon 1008 is deflated, leaving the heart valve prosthesis 300 in the radially expanded configuration within the previously implanted heart valve prosthesis 100 with the lacerate leaflets 108 separated by the gripper pads 400. In a step 1418 of the method 1400, the delivery system 1000 and the guidewire 1016 are proximally retracted and removed from the patient's vasculature, leaving the heart valve prosthesis 300 fully deployed within the previously implanted heart valve prosthesis 100 as shown in FIG. 24.

It is understood that the method 1400 described above is described using the leaflet clip mechanism 1210 shown in and described with respect to FIG. 12. However, this is not meant to be limiting, and the leaflet clip mechanism 1010 or 1110 may be used instead, with appropriate steps of the method 1400 removed. Further, other leaflet clip mechanisms may be used that can ensure engagement between the gripper pads and the valve leaflets of the previously implanted heart valve prosthesis. Further, it is understood that the heart valve prosthesis 300 of any of the embodiments shown and described herein (i.e., FIGS. 3A-3b, or FIGS. 4A-4B, or FIGS. 6A-6B) may be utilized. Further, other heart valve prostheses may be utilized provided that gripper pads are utilized therewith.

It is also understood that the description above is with respect to implanting a heart valve prosthesis within a previously implanted transcatheter heart valve prosthesis. However, this is not meant to be limiting, and a heart valve prosthesis with gripper pads may be implanted within a previously implanted surgical heart valve prosthesis, or within a native heart valve (i.e., without a previously implanted heart valve prosthesis), if it is desirable to separate lacerated leaflets of such a previously implanted surgical heart valve prosthesis native heart valve.

It will be understood that features and embodiments discussed herein can be used in combination with any other feature of any other embodiment. All patents and publications discussed herein are incorporated by reference herein in their entirety.

While only some embodiments have been described herein, it should be understood that it has been presented by way of illustration and example only, and not limitation. Various changes in form and detail can be made therein without departing from the spirit and scope of the disclosure, and each feature of the embodiment discussed herein, and of each reference cited herein, can be used in combination with the features of any other embodiment. All patents and publications discussed herein are incorporated by reference herein in their entirety.

What is claimed is:

1. A method of delivering and deploying a heart valve prosthesis, the method comprising:
advancing a delivery system including the heart valve prosthesis to a site of a native heart valve, the heart valve prosthesis including a plurality of gripper pads on an outer surface thereof;
locating the heart valve prosthesis within lacerated leaflets of the native heart valve or of a previously implanted heart valve prosthesis;
advancing a leaflet clip mechanism over the lacerated leaflets;
partially expanding the heart valve prosthesis such that a radially outward force is imparted on the gripper pads and a radially inward force is imparted on the lacerated leaflets by the leaflet clip mechanism such that the gripper pads engage with the lacerated leaflets;
proximally retracting the leaflet clip mechanism; and
fully expanding the heart valve prosthesis such that the gripper pads separate the lacerated leaflets.

2. The method of claim 1, wherein the delivery system comprises an expandable balloon, wherein the heart valve prosthesis is mounted on the balloon, wherein partially expanding the heart valve prosthesis comprises partially expanding the balloon, and wherein fully expanding heart valve prosthesis comprises further expanding the balloon.

3. The method of claim 1, wherein the leaflet clip mechanism comprises a first shaft with a plurality of fingers extending from a distal end of the first shaft, wherein advancing the leaflet clip mechanism comprises distally advancing the first shaft to distally advance the plurality of fingers.

4. The method of claim 3, wherein the plurality of fingers are configured to expand radially outward such that advancing the plurality of figures out of an outer shaft of the delivery system enables the plurality of fingers to expand radially outward and over the lacerated leaflets.

5. The method of claim 3, wherein the leaflet clip mechanism further comprises a second shaft disposed over the first shaft and a tension loop extending from a distal end of the second shaft, wherein advancing the leaflet clip mechanism further includes advancing the second shaft such that the tension loop imparts a radially inward force to the fingers to impart a radially inward force on the lacerated leaflets.

6. The method of claim 1, further comprising rotationally aligning the heart valve prosthesis relative to the native heart valve or the previously implanted heart valve prosthesis, such that the plurality of gripper pads align with the lacerated leaflets.

7. The method of claim 1, wherein locating the heart valve prosthesis comprises locating the heart valve prosthesis within lacerated leaflets of the native heart valve.

8. The method of claim 1, wherein locating the heart valve prosthesis comprises locating the heart valve prosthesis within lacerated leaflets of the previously implanted heart valve prosthesis.

9. A heart valve prosthesis having a radially expanded configuration and a radially compressed configuration, the heart valve prosthesis comprising:
a frame, the frame defining a central lumen;
a valve assembly disposed within the central lumen of the frame; and
a plurality of gripper pads, wherein each gripper pad is configured to engage lacerated leaflets of a previously implanted heart valve prosthesis or of a native heart valve,
wherein the plurality of gripper pads are configured to separate from each other to separate the lacerated leaflets of the previously implanted heart valve prosthesis or the native heart valve when the heart valve prosthesis radially expands from the radially compressed configuration to the radially expanded configuration.

10. The heart valve prosthesis of claim 9, wherein the plurality of gripper pads are coupled to an outer surface of the frame.

11. The heart valve prosthesis of claim 9, wherein the heart valve prosthesis further comprises an outer skirt coupled to an outer surface of the frame, and wherein the plurality of gripper pads are coupled to an outer surface of the outer skirt.

12. The heart valve prosthesis of claim 9, wherein the plurality of gripper pads includes at least one gripper pad pair including a first gripper pad and a second gripper pad.

13. The heart valve prosthesis of claim 12, wherein the first gripper pad and the second gripper pad are disposed adjacent each other between a first commissure of the valve assembly and a second commissure of the valve assembly.

14. The heart valve prosthesis of claim 13, wherein the first gripper pad is spaced a first circumferential distance from the second gripper pad in the radially compressed configuration and the first gripper pad is spaced a second distance from the second gripper pad in the radially expanded configuration, wherein the second distance is greater than the first distance.

15. The heart valve prosthesis of claim 9, wherein the plurality of gripper pads are substantially parallel to a central longitudinal axis of the heart valve prosthesis.

16. The heart valve prosthesis of claim 9, wherein the plurality of gripper pads are angled with respect to a central longitudinal axis of the heart valve prosthesis.

17. A delivery system for delivering a heart valve prosthesis, the delivery system comprising:
an outer shaft including an outer shaft lumen;
an inner shaft disposed within the outer shaft lumen;
a balloon coupled to the inner shaft;
the heart valve prosthesis mounted on the expandable balloon, the heart valve prosthesis including a frame, a valve assembly, and a plurality of gripper pads disposed on an outer surface of the heart valve prosthesis; and
a leaflet clip mechanism configured to selectively impart an inward radial force on a plurality of valve leaflets of a previously implanted heart valve prosthesis or of a native heart valve such that a valve leaflet of the plurality of leaflets is engaged with a gripper pad of the plurality of gripper pads.

18. The delivery system of claim 17, wherein the leaflet clip mechanism comprises a first shaft and a plurality of fingers extending distally from the first shaft, the plurality of fingers configured to engage and outer surface of the plurality of valve leaflets of the previously implanted heart valve prosthesis or of the native heart valve.

19. The delivery system of claim 18, wherein the leaflet clip mechanism further comprises a second shaft and a tension loop extending from a distal end of the second shaft, wherein the second shaft is disposed radially outside the first shaft and the tension loop is disposed radially outside the plurality of fingers, wherein the tension loop is configured to impart a radially inward force on the plurality of fingers.

20. The delivery system of claim 17, wherein the leaflet clip mechanism comprises a shaft and a tension loop extending from a distal end of the shaft, wherein the tension loop is configured to impart a radially inward force on the plurality of valve leaflets of the previously implanted heart valve prosthesis or the native heart valve.
